SEQUENCE LISTING (18 sequences)

SEQ ID NO: 1 - DNA, 3586 bp, Mus musculus
  CDS (15)..(2903)
gcgggctgcgcaagatggcgacgacggccgcatcttcggcgtccgagccggaggttgagccccaggccgggcctgaggccgagggagaagaggatgaggcgaagccggccggtgtgcagcggaaggtgctgtccggcgctgtggccgcggaggcggcggaggccaagggccccggatgggacctgcagcgggaaggcgcgagcgggagcgatggggatgaggaggacgccatggcttcctccgccgagagctccgccggggaggacgagtgggagtacgacgaggaggaggagaagaaccagctggagatcgagcggctggaggagcagctgtccatcaatggctatgattacaactgccacgtggagctcatcaggctgctgcggctggaaggcgagctcagcagagtgagggcggcccgccagaagatgagtgagctcttccccctgaccgaagagctctggctggagtggctccacgatgagatcagcatggccatggacggcctggaccgcgagcacgtgtacgagctctttgagagagccgtgaaggactacatctgtccaaacatttggctagagtatggccagtactcagttggtggcattggtcagaaaggtggccttgagaaggttcgctctgtctttgaaagagccctgtcctctgttggcctgcacatgacgaaaggcctggccatctgggaggcctaccgagagtttgaaagcgccatcgtggaggctgctcggctggagaaagtgcacagtctcttccggcgacagctggcgatcccactgtacgagatggaggccacctttgcagagtatgaagaatggtcagaggagcccatgccggagtctgtacttcagagctatcagaaagcgctggggcagctagagaagtacaagccttacgaggaagcgctgctgcaggcagaggcccctcggctggcggaataccaagcttacatcgacttcgagatgaaaatcggggatcctgcccgtattcagttgatctttgagcgtgccctggtggagaactgcctggttccagacttatggatccgctacagtcagtacctagatcgacagctgaaagttaaggacttggttttatctgtacacagccgtgctgtgcggaactgcccatggacagttgccctgtggagtcggtaccttctggccatggagcgacatggactggaccatcaaacgatttctgcgaccttcgagaacgctctgagtgccggcttcatccaggccactgactatgtggagatctggcaggtgtacctcgactacctgaggagaagggttgacttcagacaggactctagcaaggagctggaagagctgcggtccatgttcacgcgagctctggagtacctgcagcaggaggttgaggagcgtttcagcgagagtggggatccaagctgcctgatcatgcagagctgggctcgggttgaggctcgcctgtgcaataacatgcagaaagcccgagagctctgggacagcatcatgaccagagggaatgccaagtacgccaacatgtggctggagtattacaacctggaacgggcacacggtgacacacaacactgtcggaaggctctgcaccgagctgtccagtgcacgagtgactaccctgagcacgtctgtgaagtgttgctcaccatggagaggacagaagggaccttagaagattgggatctagccattcagaaaacggagacgcgcttggctcgtgtgaatgagcagagaatgaaggccgcagagaaggaagcagctcttgtgcagcaggaagaagaaaaggccgagcagcggaagaaggtgcgggcggagaagaaagccctgaaaaagaagaagaaaacgcgaggtgccgacaagcgcagggaggacgaggacgaggagaacgagtggggcgaagaggaggaagagcagccttccaaacgcagaaggacggagaacagtctggcctctggagaggcttcggctatgaaggaagaaacagagctctccgggaaatgcttaacgatagatgttggtcctccttccaagcagaaagagaaggcagcctcccttaagcgggacatgcccaaggtggctcacgacagcagtaaggacagtgtcaccgtgtttgtcagcaacctgccctacagcatagaagagcccgaggtgaagctcaggccgctctttgaggtctgtggggaggtggtccagatcaggccaattttcagcaaccgcggggacttccggggctactgctatgtggagtttggagaggagaagtcagcccagcaggccctggagctggacaggaagattgtggagggcaggccgatgtttgtgtccccctgtgtggataagagcaaaaaccctgattttaaggtgttcagatacagtaccaccctggagaaacacaaactcttcatctctggcctgcccttttcctgcaccaaagaggagctcgaggacatttgtaaggcccacggcaccgtcaaggacctcaggctggtcactaacagggctggcaagccgaagggcctggcgtatgtggagtatgaaaacgagtcccaggcgtcccaggcagtgatgaagatggacggcatgaccatcagagagaatgtcatcaaggtggcaatcagcaatccccctcagcgaaaagtcccagagaagccagaagtgaggacagcaccaggggcccccatgctcccccggcagatgtatggcgcgcgcgggaagggacggacccagctctctcttcttcctcgagctctgcagcgccagggtgctgctcctcaggctgagaacggcccagctccggggcccgcggtcgccccgtctgtggccacagaggctcctaagatgtccaatgctgattttgcgaagttgcttctgagaaagtgagcaggactctgagatggagatgccttacctgtcctcaagctggccgggctggccaccacgggccctggagacggaagggctgggcacttgcctgcgctcccacagattctcctctggtgtggatgggaagggagagcctatggtgaacatggcggtgaggagtgttccctcacattgagggcggaggccaaccgctctacaggctgtcccaaggtacgttagtgtcctaacaaggagggacccagctttcgaggcccacttgtcctgatgctttcaccgcctctggccccttttctacgaaccccctcccccagccctgcacagcacgtgtgcccatcactctgtaagtgtggaagatggaatgggagagcttgtcactcatcagaatggcctgtcgagaagtgcgggacgtcacagaagacacgtgtgatgggcttttgtccaaagaggctatgagtttttctattatgtatttctaattgacactgtttaatgttccctaaaagtggaatgtctgcgacctttgttcttaggaattgtgtgccagccactctggaccactttctttgcctgatgactggagccctaagccctctgttcagatgctcacttcgaaatgccatgtctagtacaaagttgagtctccccattgagttttgtttattaaactgaagttcttacag

SEQ ID NO: 2 - protein, 962 aa, Mus musculus
MATTAASSASEPEVEPQAGPEAEGEEDEAKPAGVQRKVLSGAVAAEAAEAKGPGWDLQREGASGSDGDEEDAMASSAESSAGEDEWEYDEEEEKNQLEIERLEEQLSINGYDYNCHVELIRLLRLEGELSRVRAARQKMSELFPLTEELWLEWLHDEISMAMDGLDREHVYELFERAVKDYICPNIWLEYGQYSVGGIGQKGGLEKVRSVFERALSSVGLHMTKGLAIWEAYREFESAIVEAARLEKVHSLFRRQLAIPLYEMEATFAEYEEWSEEPMPESVLQSYQKALGQLEKYKPYEEALLQAEAPRLAEYQAYIDFEMKIGDPARIQLIFERALVENCLVPDLWIRYSQYLDRQLKVKDLVLSVHSRAVRNCPWTVALWSRYLLAMERHGLDHQTISATFENALSAGFIQATDYVEIWQVYLDYLRRRVDFRQDSSKELEELRSMFTRALEYLQQEVEERFSESGDPSCLIMQSWARVEARLCNNMQKARELWDSIMTRGNAKYANMWLEYYNLERAHGDTQHCRKALHRAVQCTSDYPEHVCEVLLTMERTEGTLEDWDLAIQKTETRLARVNEQRMKAAEKEAALVQQEEEKAEQRKKVRAEKKALKKKKKTRGADKRREDEDEENEWGEEEEEQPSKRRRTENSLASGEASAMKEETELSGKCLTIDVGPPSKQKEKAASLKRDMPKVAHDSSKDSVTVFVSNLPYSIEEPEVKLRPLFEVCGEVVQIRPIFSNRGDFRGYCYVEFGEEKSAQQALELDRKIVEGRPMFVSPCVDKSKNPDFKVFRYSTTLEKHKLFISGLPFSCTKEELEDICKAHGTVKDLRLVTNRAGKPKGLAYVEYENESQASQAVMKMDGMTIRENVIKVAISNPPQRKVPEKPEVRTAPGAPMLPRQMYGARGKGRTQLSLLPRALQRQGAAPQAENGPAPGPAVAPSVATEAPKMSNADFAKLLLRK

SEQ ID NO: 3 - DNA, 1561 bp, Homo sapiens
  CDS (172)..(1146)
gggcttatcccgcctgtcccgccattctcgctagttcgatcggtagcgggagcggagagcggaccccagagagccctgagcagccccaccgccgccgccggcctagttaccatcacaccccgggaggagccgcagctgccgcagccggccccagtcaccatcaccgcaaccatgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgcggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcgtccgctcccgaggctgagcagggcggggctgagtaaatgccggcttaccatctctaccatcatccggtttagtcatccaacaagaagaaatatgaaattccagcaataagaaatgaacaaaagattggagctgaagacctaaagtgcttgctttttgcccgttgaccagataaatagaactatctgcattatctatgcagcatggggtttttattatttttacctaaagacgtctctttttggtaataacaaacgtgttttttaaaaaagcctggtttttctcaatacgcctttaaaggtttttaaattgtttcatatctggtcaagttgagatttttaagaacttcatttttaatttgtaataaaagtttacaacttgattttttcaaaaaagtcaacaaactgcaagcacctgttaataaaggtcttaaataataaaaaaaaaaaaaaa

SEQ ID NO: 4 - protein, 324 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 5 - DNA, 2243 bp, Mus musculus
  CDS (65)..(1960)
ctcacacacggagcgccagccttgagtttgttttctagccccttcccgcctgttcaccaccaccatgaccccgggcattcgggctcctttcttcctgctgctacttctagcaagtctaaaaggttttcttgcccttccaagtgaggaaaacagtgtcacctcatctcaggacaccagcagttccttagcatcgactaccactccagtccacagcagcaactcagacccagccaccagacctccaggggactccaccagctctccagtccagagtagcacctcttctccagccaccagagctcctgaagactctaccagtactgcagtcctcagtggcacctcctccccagccaccacagctccagtgaactccgccagctctccagtagcccatggtgacacctcttccccagccactagcctttcaaaagactccaacagctctccagtagtccacagtggcacctcttcagctccggccaccacagctccagtggattccaccagctctccagtagtccacggtggtacctcgtccccagccaccagccctccaggggactccaccagctctccagaccatagtagcacctcttctccagccaccagagctcccgaagactctaccagtactgcagtcctcagtggcacctcctccccagccaccacagctccagtggactccaccagctctccagtagcccatgatgacacctcttccccagccactagcctttcagaagactccgccagctctccagtagcccacggtggcacctcttctccagccaccagccctctaagggactccaccagttctccagtccacagtagtgcctccatccaaaacatcaagactacatcagacttagctagcactccagaccacaatggcacctcagtcacaactaccagctctgcactgggctcagccaccagtccagaccacagtggtacctcaactacaactaacagctctgaatcagtcttggccaccactccagtttacagtagcatgccattctctactaccaaagtgacgtcaggctcagctatcattccagaccacaatggctcctcggtgctacctaccagttctgtgttgggctcagctaccagtctagtctataatacctctgcaatagctacaactccagtcagcaatggcactcagccttcagtgccaagtcaataccctgtttctcctaccatggccaccacctccagccacagcactattgccagcagctcttactatagcacagtaccattttctaccttctccagtaacagttcaccccagttgtctgttggggtctccttcttcttcttgtctttttacattcaaaaccacccatttaattcttctctggaagaccccagctccaactactaccaagaactgaagaggaacatttctggattgtttctgcagatttttaacggagattttctggggatctctagcatcaagttcaggtcaggctccgtggtggtagaatcgactgtggttttccgggagggtacttttagtgcctctgacgtgaagtcacagcttatacagcataagaaggaggcagatgactataatctgactatttcagaagtcaaagtgaatgagatgcagttccctccctctgcccagtcccggccgggggtaccaggctggggcattgccctgctggtgctggtctgtattttggttgctttggctatcgtctatttccttgccctggcagtgtgccagtgccgccgaaagagctatgggcagctggacatctttccaacccaggacacctaccatcctatgagtgaataccctacctaccacactcacggacgctacgtgccccctggcagtaccaagcgtagcccctatgaggaggtttcggcaggtaatggcagtagcagtctctcttataccaacccagctgtggtgaccacttctgccaacttgtaggagcaagtcaccccacccacttggggcagctttggcggtctgctccctcagtggtcactgccagacccctgcactctgatctgggctggtgagccaggacttctggtaggctgttcatgccctttgtcaagcgcctcaactacgtaagcctggtgaagcccagccctgccctgggggacactggggcagttagtggtggctctcagaaggactggcctggaaaactggagacagggatgggaacccaaacatagctgaataaaagatggcctcctgttagtta

SEQ ID NO: 6 - protein, 631 aa, Mus musculus
MTPGIRAPFFLLLLLASLKGFLALPSEENSVTSSQDTSSSLASTTTPVHSSNSDPATRPPGDSTSSPVQSSTSSPATRAPEDSTSTAVLSGTSSPATTAPVNSASSPVAHGDTSSPATSLSKDSNSSPVVHSGTSSAPATTAPVDSTSSPVVHGGTSSPATSPPGDSTSSPDHSSTSSPATRAPEDSTSTAVLSGTSSPATTAPVDSTSSPVAHDDTSSPATSLSEDSASSPVAHGGTSSPATSPLRDSTSSPVHSSASIQNIKTTSDLASTPDHNGTSVTTTSSALGSATSPDHSGTSTTTNSSESVLATTPVYSSMPFSTTKVTSGSAIIPDHNGSSVLPTSSVLGSATSLVYNTSAIATTPVSNGTQPSVPSQYPVSPTMATTSSHSTIASSSYYSTVPFSTFSSNSSPQLSVGVSFFFLSFYIQNHPFNSSLEDPSSNYYQELKRNISGLFLQIFNGDFLGISSIKFRSGSVVVESTVVFREGTFSASDVKSQLIQHKKEADDYNLTISEVKVNEMQFPPSAQSRPGVPGWGIALLVLVCILVALAIVYFLALAVCQCRRKSYGQLDIFPTQDTYHPMSEYPTYHTHGRYVPPGSTKRSPYEEVSAGNGSSSLSYTNPAVVTTSANL

SEQ ID NO: 7 - DNA, 924 bp, Mus musculus
  CDS (16)..(438)
tgtggtgacgccatcatgggagctccggcgctgccccagatctggcagctgtacctcaagaactaccgcatcgccaccttcaagaactggcccttcctggaggactgcgcctgcaccccagagcgaatggcggaggctggcttcatccactgccctaccgagaacgagcctgatttggcccagtgttttttctgctttaaggaattggaaggctgggaacccgatgacaacccgatagaggagcatagaaagcactcccctggctgcgccttcctcactgtcaagaagcagatggaagaactaaccgtcagtgaattcttgaaactggacagacagagagccaagaacaaaattgcaaaggagaccaacaacaagcaaaaagagtttgaagagactgcaaagactacccgtcagtcaattgagcagctggctgcctaatgctgagcctttgctgagataacttggacctgagtgacatgccacatctaagccacgcatcccagcttttccagccagggcctcctagcaggatcttagagaaggagacagtggtattttgaaactggatatcaaatatttttggttttgctttaaagtggctacctctctttggttttgtggctttgctctattgtgacgtggacttaagcaataaggaagtgatgaagggacagtgttctctgacaggacctgtgggggtcggggtgcctgtgcaaggtcttggttctgattgtgatatttccatacagggctgctaatgcagcccatgggtaagtgtggttatatgtgtttgtgctgataattttgtcctgatgagttttcctaccacggggtaacggaataaaatcacttgaaaaagtggactgtaaaaaaaaaaaaaaaaaaaaaaaaaaaaaaacaaaaaaaaaaaaaaaaaaaaaaaaa

SEQ ID NO: 8 - protein, 140 aa, Mus musculus
MGAPALPQIWQLYLKNYRIATFKNWPFLEDCACTPERMAEAGFIHCPTENEPDLAQCFFCFKELEGWEPDDNPIEEHRKHSPGCAFLTVKKQMEELTVSEFLKLDRQRAKNKIAKETNNKQKEFEETAKTTRQSIEQLAA

SEQ ID NO: 9 - DNA, 1033 bp, Mus musculus
  CDS (290)..(715)
ggtcagactgcccaggcagggtgggaaaggcctttaaagcagcccgcaggtgggctgccagttcttggaagggcttattaatgaaaaccccccaagcctgacaacctgggggaaggctcactggccccatgtatagctgataagggccaggagattccacaactcaggtagttcccccgcccccctggagttctgtggtcaccattaatcatttcctctaactgtgtatataagagctcttttgcagtgagcccagtactcagagagaaaggctaaggtcctgaggaggatgtggctgcagaatttacttttcctgggcattgtggtctacagcctctcagcacccacccgctcacccatcactgtcacccggccttggaagcatgtagaggccatcaaagaagccctgaacctcctggatgacatgcctgtcacgttgaatgaagaggtagaagtcgtctctaacgagttctccttcaagaagctaacatgtgtgcagacccgcctgaagatattcgagcagggtctacggggcaatttcaccaaactcaagggcgccttgaacatgacagccagctactaccagacatactgccccccaactccggaaacggactgtgaaacacaagttaccacctatgcggatttcatagacagccttaaaacctttctgactgatatcccctttgaatgcaaaaaaccaggccaaaaatgaggaagcccaggccagctctgaatccagcttctcagactgctgcttttgtgcctgcgtaatgagccaggaacttggaatttctgccttaaagggaccaagagatgtggcacagccacagttggaaggcagtatagccctctgaaaacgctgactcagcttggacagcggaagacaaacgagagatattttctactgatagggaccattatatttatttatatatttatattttttaaatatttatttatttatttatttatttttgcaactctatttattgagaatgtcttaccagaataataaattattaaaactttt

SEQ ID NO: 10 - protein, 141 aa, Mus musculus
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 11 - DNA, 1250 bp, Mus musculus
  CDS (13)..(795)
ctttcagtcagcatgatagaaacatacagccaaccttcccccagatccgtggcaactggacttccagcgagcatgaagatttttatgtatttacttactgttttccttatcacccaaatgattggatctgtgctttttgctgtgtatcttcatagaagattggataaggtcgaagaggaagtaaaccttcatgaagattttgtattcataaaaaagctaaagagatgcaacaaaggagaaggatctttatccttgctgaactgtgaggagatgagaaggcaatttgaagaccttgtcaaggatataacgttaaacaaagaagagaaaaaagaaaacagctttgaaatgcaaagaggtgatgaggatcctcaaattgcagcacacgttgtaagcgaagccaacagtaatgcagcatccgttctacagtgggccaagaaaggatattataccatgaaaagcaacttggtaatgcttgaaaatgggaaacagctgacggttaaaagagaaggactctattatgtctacactcaagtcaccttctgctctaatcgggagccttcgagtcaacgcccattcatcgtcggcctctggctgaagcccagcagtggatctgagagaatcttactcaaggcggcaaatacccacagttcctcccagctttgcgagcagcagtctgttcacttgggcggagtgtttgaattacaagctggtgcttctgtgtttgtcaacgtgactgaagcaagccaagtgatccacagagttggcttctcatcttttggcttactcaaactctgaacagtgcgctgtcctaggctgcagcagggctgatgctggcagtcttccctatacagcaagtcagttaggacctgccctgtgttgaactgcctatttataaccctaggatcctcctcatggagaactatttattatgtacccccaaggcacatagagctggaataagagaattacagggcaggcaaaaatcccaagggaccctgctccctaagaacttacaatctgaaacagcaaccccactgattcagacaaccagaaaagacaaagccataatacacagatgacagagctctgatgaaacaacagataactaatgagcacagttttgttgttttatgggtgtgtcgttcaatggacagtgtacttgacttaccagggaagatgcagaagggcaactgtgagcctcagctcacaatctgttatggttgacctgggctccctgcggccctagtagg

SEQ ID NO: 12 - protein, 260 aa, Mus musculus
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 13 - DNA, 1512 bp, Artificial Sequence
  Synthetic DNA
ggatccatgggcctgagcaacatcctgttcgtgatggcctttctgctgagcggcgccgccccactgaagatccaggcctactttaacgacatcgtgctgactcagtctcctgcctctctggccgtgtctctgggacagagagccacaatcagctgcagagccagcgagagcgtggagtactacgtgaccagcctgatgcagtggtaccagcagaagcccggccagccacccaagctgctgatttacgccgccagcaacgtggagagcggcgtgccagccagattttctggcagcggctctggcaccgatttcagcctgaacatccaccccgtggaggaggacgacatcgccatgtacttctgccagcagaccagaaaggtgcccagcatcttcggcggcggcacaaagctggagattaaaagagctggaggaggcggatctggaggaggaggctctgggggggggggctcccaggtgcagctgaaagaaagcggcccaggcctggtggccccatctcagagcctgagcattacctgcaccgtgagcggcttcagcctgaccagctatggcgtgcattgggtgagacagcctcccggaaaaggcctggaatggctgggcgtgatttgggccggcggcagcaccaattacaacagcgccctgatgagcagactgagcatcagcaaggacaacagcaagagccaggtgttcctgaagatgaacagcctgcagaccgacgataccgccatgtattattgtgccagagacaagagagcccccggcaagctgtactacggctaccccgattattggggccagggcacaactctgacagtgtctagtggcggcggaggcagccacaagaagcctaccggcatgatcagaatccaccagatgaacagcgagctgagcgtgctggccaacttcagccagcccgagatcgtgcccatcagcaacatcaccgagaacgtgtacatcaacctgacctgcagcagcatccacggctaccccgagcccaagaagatgagcgtgctgctgagaaccaagaacagcaccatcgagtacgacggcatcatgcagaagagccaggacaacgtgaccgagctgtacgacgtgagcatcagcctgagcgtgagcttccccgacgtgaccagcaacatgaccatcttctgcatcctggagaccgacaagaccagactgctgagcagcccctttagcatcgagctggaggacccccagccccctcccgatcacatcccatggatcaccgccgtgctgcctaccgtgatcatctgcgtgatggtgttctgcctgatcctgtggaagtggaagaagaagaagagacccagaaacagctacaagtgcggcaccaacaccatggagagagaggagagcgagcagaccaagaagagagagaagatccacatccccgagagaagcgacgaggcccagagagtgttcaagagcagcaagaccagcagctgcgacaagagcgacacctgcttctgaaagctt

SEQ ID NO: 14 - protein, 499 aa, Artificial Sequence
  Synthetic Construct
MGLSNILFVMAFLLSGAAPLKIQAYFNDIVLTQSPASLAVSLGQRATISCRASESVEYYVTSLMQWYQQKPGQPPKLLIYAASNVESGVPARFSGSGSGTDFSLNIHPVEEDDIAMYFCQQTRKVPSIFGGGTKLEIKRAGGGGSGGGGSGGGGSQVQLKESGPGLVAPSQSLSITCTVSGFSLTSYGVHWVRQPPGKGLEWLGVIWAGGSTNYNSALMSRLSISKDNSKSQVFLKMNSLQTDDTAMYYCARDKRAPGKLYYGYPDYWGQGTTLTVSSGGGGSHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 15 - DNA, 21 bp, Artificial Sequence
  Synthetic nucleic acid
cuacagucaguaccuagautt

SEQ ID NO: 16 - DNA, 21 bp, Artificial Sequence
  Synthetic nucleic acid
aucuagguacugacuguagtt

SEQ ID NO: 17 - DNA, 19 bp, Artificial Sequence
  Synthetic nucleic acid
gtgagctcttccccctgac

SEQ ID NO: 18 - DNA, 20 bp, Artificial Sequence
  Synthetic nucleic acid
catgctgatctcatcgtgga

(19) United States
(12) Patent Application Publication
(10) Pub. No.: US 2016/0058856 A1
(43) Pub. Date: Mar. 3, 2016

(54) ANTI-TUMOR DNA VACCINE

(71) Applicant: KYUSHU UNVERSITY, NATIONAL UNIVERSITY CORPORATION, Fukoka-shi, Fukuoka (JP)

(72) Inventor: Kenji NAKANO, Fukuoka-shi (JP)

(73) Assignee: KYUSHU UNIVERSITY, NATIONAL UNIVERSITY CORPORATION, Fukuoka-shi, Fukuoka (JP)

(21) Appl. No.: 14/781,609

(22) PCT Filed: Apr. 4, 2014
(86) PCT No.: PCT/JP2014/060356

§ 371 Date: Oct. 1, 2015

(30) Foreign Application Priority Data
Apr. 5, 2013 (JP) 2013-079854

(51) Int. Cl. A61K 39/00; A61K 39/39

(52) U.S. Cl. CPC A61K 39/0011; A61K 39/39; A61K 2039/55561; A61K 2039/53; A61K 2039/55522; A61K 2039/55516; A61K 2039/55555

(57) ABSTRACT

The present invention provides a pharmaceutical composition for treating a tumor, which is a micelle encapsulating at least one tumor-associated antigen gene. The present invention also provides a method for treating a tumor, comprising administering a micelle encapsulating at least one tumor-associated antigen gene to a patient in need of such treatment.

### RELATED ART

The present invention relates to a pharmaceutical composition for treating a tumor, which is a gene carrier device, micelle encapsulating at least one tumor-associated antigen gene. The present invention also relates to a method for treating a tumor, comprising administering a micelle encapsulating at least one tumor-associated antigen gene to a subject in need of such treatment.

### BACKGROUND ART

Cancer vaccines have attracted much attention as a promising modality to treat patients with malignancies as they induce potent anti-tumor effects with reduced invasiveness in contrast to chemo-, irradiation- and surgical therapies. The anti-tumor effect is mediated by the activation of tumor-specific rejection immunity. Tumor-associated antigen (TAA) is delivered into dendritic cells (DC)/antigen-presenting cells (APC) [1] where fragmented TAA-peptides are expressed by major histocompatibility antigen complex (MHC) class-1 and -2 molecules on the cell surface. These are recognized by specific cytotoxic and helper T lymphocytes, respectively, which become activated in concert with co-stimulatory interactions such as B7/CD28 and CD40/CD40L [2]. Extracellular stimuli by granulocyte macrophage colony-stimulating factor (GM-CSF) matures DC/APC cells to upregulate MHC class-2 expression [3], resulting in an enhanced vaccination effect [4].

Three types of peptide-, cell- and gene-based vaccines have been investigated in basic research and clinical trials for cancer treatment. Peptide vaccines have the properties of low production cost, high safety and good compliance in clinical application; however, it is difficult to identify which TAA-epitope peptides elicit strong vaccination effects against tumors with relative low immunogenicity [5, 6]. It is also necessary to match between epitope-peptide and MHC type, resulting in a limited eligibility of patients receiving peptide vaccines [5, 6]. For cell vaccines, viral vectors are usually used to transduce TAA-genes into cultured DC or autologous tumor cells. Cell-based vaccines are time-consuming, less versatile, have safety issues regarding pathogens, and have a high production cost [7]. However, gene-based vaccines could resolve these issues if anti-tumor immunity is vigorously elicited by transduction of TAA alone or with the addition of adjuvant genes without viral vectors [8].

Non-viral gene carrier devices have been extensively studied using various materials, such as cationic liposomes [9, 10], polysaccharides [11, 12], dendrimers [13, 14] and polycatiomers [15-17]. Nevertheless, these synthetic carriers have limited transduction efficiency without causing normal tissue injury in vivo. Recently, extended modifications to polycatiomers have improved polyplex-based gene carriers to achieve gene transduction with minimum injury of normal organs in vivo [18-21].

### REFERENCES

1. Smits, E L, Anguille, S, Cools, N, Berneman, Z N, and Van Tendeloo, V F (2009). Dendritic cell-based cancer gene therapy. Human gene therapy 20: 1106-1118.
2. Andersen, B M, and Ohlfest, J R (2012). Increasing the efficacy of tumor cell vaccines by enhancing cross priming. Cancer letters 325: 155-164.
3. Lu, L, et al. (1994). Propagation of dendritic cell progenitors from normal mouse liver using granulocyte/macrophage colony-stimulating factor and their maturational development in the presence of type-1 collagen. The Journal of experimental medicine 179: 1823-1834.
4. van de Laar, L, Coffer, P J, and Woltman, A M (2012). Regulation of dendritic cell development by GM-CSF: molecular control and implications for immune homeostasis and therapy. Blood 119: 3383-3393.
5. Lazoura, E, and Apostolopoulos, V (2005). Insights into peptide-based vaccine design for cancer immunotherapy. Current medicinal chemistry 12: 1481-1494.
6. Berzofsky, J A, Terabe, M, and Wood, L V (2012). Strategies to use immune modulators in therapeutic vaccines against cancer. Seminars in oncology 39: 348-357.
7. Mackiewicz, J, and Mackiewicz, A (2009). Design of clinical trials for therapeutic cancer vaccines development. European journal of pharmacology 625: 84-89.
8. van den Berg, J H, Oosterhuis, K, Beijnen, J H, Nuijen, B, and Haanen, J B (2010). DNA vaccination in oncology: current status, opportunities and perspectives. Current clinical pharmacology 5: 218-225.
9. Liu, Y, et al. (1997). Factors influencing the efficiency of cationic liposome-mediated intravenous gene delivery. Nature biotechnology 15: 167-173.
10. Schafer, J, Hobel, S, Bakowsky, U, and Aigner, A (2010). Liposome-polyethylenimine complexes for enhanced DNA and siRNA delivery. Biomaterials 31: 6892-6900.
11. Du, Y Z, Lu, P, Zhou, J P, Yuan, H, and Hu, F Q (2010). Stearic acid grafted chitosan oligosaccharide micelle as a promising vector for gene delivery system: factors affecting the complexation. International journal of pharmaceutics 391: 260-266.
12. Beaudette, T T, et al. (2009). Chemoselective ligation in the functionalization of polysaccharide-based particles. Journal of the American Chemical Society 131: 10360-10361.
13. Liu, H, Wang, H, Yang, W, and Cheng, Y (2012). Disulfide cross-linked low generation dendrimers with high gene transfection efficacy, low cytotoxicity, and low cost. Journal of the American Chemical Society 134: 17680-17687.
14. Nam, H Y, Nam, K, Lee, M, Kim, S W, and Bull, D A (2012). Dendrimer type bio-reducible polymer for efficient gene delivery. Journal of controlled release: official journal of the Controlled Release Society 160: 592-600.
15. Howard, K A, et al. (2004). Formulation of a microparticle carrier for oral polyplex-based DNA vaccines. Biochimica et biophysica acta 1674: 149-157.
16. Miyata, K, et al. (2008). Polyplexes from poly(aspartamide) bearing 1,2-diaminoethane side chains induce pH-selective, endosomal membrane destabilization with amplified transfection and negligible cytotoxicity. Journal of the American Chemical Society 130: 16287-16294.
17. Takae, S, et al. (2008). PEG-detachable polyplex micelles based on disulfide-linked block catiomers as bioresponsive nonviral gene vectors. Journal of the American Chemical Society 130: 6001-6009.
18. Harada-Shiba, M, et al. (2009). Intratracheal gene transfer of adrenomedullin using polyplex nanomicelles attenuates monocrotaline-induced pulmonary hypertension in rats. Molecular therapy: the journal of the American Society of Gene Therapy 17: 1180-1186.
19. Itaka, K, et al. (2007). Bone regeneration by regulated in vivo gene transfer using biocompatible polyplex nanomicelles. Molecular therapy: the journal of the American Society of Gene Therapy 15: 1655-1662.
20. Vachutinsky, Y, et al. (2011). Antiangiogenic gene therapy of experimental pancreatic tumor by sFlt-1 plasmid DNA carried by RGD-modified crosslinked polyplex micelles. Journal of controlled release: official journal of the Controlled Release Society 149: 51-57.
21. Itaka, K, Osada, K, Morii, K, Kim, P, Yun, S H, and Kataoka, K (2010). Polyplex nanomicelle promotes hydrodynamic gene introduction to skeletal muscle. Journal of controlled release: official journal of the Controlled Release Society 143: 112-119.

### DISCLOSURE OF THE INVENTION

Gene carrier micelle has been recently demonstrated to achieve efficient gene transduction and biocompatibility in vivo. In the present study, we investigated the potential as a DNA vaccine platform of micelle encapsulating tumor-associated antigen (TAA), CD40L and GM-CSF genes via intraperitoneal (i.p.) administration in mouse tumor models. The DNA vaccine with TAA (SART3 or YB-1), CD40L and GM-CSF genes significantly prolonged the survival for the mice harboring colon-26 peritoneal dissemination compared with the mock control, or single gene therapy. The re-challenge experiment confirmed that long-period survivor mice treated with the DNA vaccine gained the rejection memory immunity. The DNA vaccine also inhibited the growth and lung metastasis in subcutaneous tumors of colon-26 and Lewis lung cancers. In both tumor models, the cytotoxic T cells (CTL) activity was highly elicited only by the DNA vaccine, while the NK activity was induced by micelles with GM-CSF transgene. The specificity to major histocompatibility antigen complex and SART3 molecules in the CTL activity was confirmed using blocking anti-MHC antibodies and SART3 siRNA knockdown. Furthermore, the infiltration of GM-CSF and CD11c-positive cells in lymph nodes and spleen on day 7, and that of CD4 and CD8a-positive T lymphocytes into subcutaneous tumors on days 14 and 28 was enhanced by the DNA vaccine treatment. These data indicate that the TAA/CD40L/GM-CSF genes-loading micelle is a novel vaccine platform to elicit CTL-mediated rejection immunity and eradicate tumor growth and metastasis.

As such, the present invention provides the followings:
[1] A pharmaceutical composition for treating a tumor, which is a micelle encapsulating at least one tumor-associated antigen gene and at least one adjuvant gene.
[2] The pharmaceutical composition of [1], wherein the tumor-associated antigen gene is at least one selected from the group consisting of squamous cell carcinoma antigen recognized by T cells 3 (SART3), Y-box binding protein 1 (YB-1), Mucin 1, cell surface associated (MUC1) and Survivin.
[3] The pharmaceutical composition of [1] or [2], wherein the adjuvant gene is at least one selected from the group consisting of Granulocyte-macrophage colony-stimulating factor (GM-CSF) and CD40L.
[4] The pharmaceutical composition according to any one of [1] to [3], wherein the adjuvant gene is any one of polynucleotide selected from the group consisting of (a) to (e) below:

(a) a polynucleotide comprising the nucleotide sequence of SEQ ID NO: 13;

(b) a polynucleotide encoding a protein consisting of the amino acid sequence of SEQ ID NO: 14

(c) a polynucleotide encoding a protein consisting of an amino acid sequence wherein 1 to 40 amino acids are deleted, substituted, inserted and/or added in the amino acid sequence of SEQ ID NO: 14, and having an activity of 28scFv(LH)-CD86 chimera;

(d) a polynucleotide encoding a protein having an amino acid sequence having at least 85% homology to the amino acid sequence of SEQ ID NO: 14, and having an activity of 28scFv(LH)-CD86 chimera; and,

(e) a polynucleotide which hybridizes to a polynucleotide consisting of a nucleotide sequence complementary to the nucleotide sequence of SEQ ID NO: 13 under stringent conditions, and which encodes a protein having an activity of 28scFv(LH)-CD86 chimera.
[5] The pharmaceutical composition of [4], comprising the polynucleotide in combination with any one or both of both of GM-CSF and CD40L.
[6] The pharmaceutical composition any one of [1] to [5], wherein the micelle is a polyion complex micelle.
[7] The pharmaceutical composition according to any one of [1] to [6], wherein the tumor is one selected from the group consisting of osteosarcoma, soft tissue sarcoma, carcinoma of the breast, carcinoma of the lung, carcinoma of the bladder, carcinoma of the thyroid gland, carcinoma of the prostate, carcinoma of the colon, colorectal carcinoma, carcinoma of the pancreas, carcinoma of the stomach, carcinoma of the liver, carcinoma of the uterus, carcinoma of the cervix, carcinoma of the ovary, Hodgkin lymphoma, non-Hodgkin lymphoma, neuroblastomas, melanomas, myelomas, Wilms tumors, acute myelocytic leukemia (AML), chronic myelocytic leukemia (CML), acute lymphocytic leukemia (ALL), chronic lymphocytic leukemia (CLL), gliomas, and retinoblastomas.
[8] A method for preventing and/or treating a tumor in a subject, comprising administering an effective amount of a micelle encapsulating at least one tumor-associated antigen gene and at least one adjuvant gene to the subject.
[9] The method according to [8], wherein the tumor is prevented by acquired rejection memory immunity.
[10] The method according to [8] or [9], wherein the tumor-associated antigen gene is at least one selected from the group consisting of squamous cell carcinoma antigen recognized by T cells 3 (SART3), Y-box binding protein 1 (YB-1), Mucin 1, cell surface associated (MUC1), and Survivin
[11] The method according to any one of [8] to [10], wherein the adjuvant gene is at least one selected from the group consisting of Granulocyte-macrophage colony-stimulating factor (GM-CSF) and CD40L.
[12] The method of according to any one of [8] to [10], wherein the adjuvant gene is any one of polynucleotide selected from the group consisting of (a) to (e) below:

(a) a polynucleotide comprising the nucleotide sequence of SEQ ID NO: 13;

(b) a polynucleotide encoding a protein consisting of the amino acid sequence of SEQ ID NO: 14

(c) a polynucleotide encoding a protein consisting of an amino acid sequence wherein 1 to 40 amino acids are deleted, substituted, inserted and/or added in the amino acid sequence of SEQ ID NO: 14, and having an activity of 28scFv(LH)-CD86 chimera;

(d) a polynucleotide encoding a protein having an amino acid sequence having at least 85% homology to the amino acid sequence of SEQ ID NO: 14, and having an activity of 28scFv(LH)-CD86 chimera; and,

(e) a polynucleotide which hybridizes to a polynucleotide consisting of a nucleotide sequence complementary to the nucleotide sequence of SEQ ID NO: 13 under stringent conditions, and which encodes a protein having an activity of 28scFv(LH)-CD86 chimera.
[13] The method according to [12], wherein said polynucleotide may be used in combination with any one or both of GM-CSF and CD40L.
[14] The method according to any one of [8] to [13], wherein the micelle is a polyion complex micelle.
[15] The method according to any one of [8] to [14], wherein the tumor is one selected from the group consisting of osteosarcoma, soft tissue sarcoma, carcinoma of the breast, carcinoma of the lung, carcinoma of the bladder, carcinoma of the thyroid gland, carcinoma of the prostate, carcinoma of the colon, colorectal carcinoma, carcinoma of the pancreas, carcinoma of the stomach, carcinoma of the liver, carcinoma of the uterus, carcinoma of the cervix, carcinoma of the ovary, Hodgkin lymphoma, non-Hodgkin lymphoma, neuroblastomas, melanomas, myelomas, Wilms tumors, acute myelocytic leukemia (AML), chronic myelocytic leukemia (CML), acute lymphocytic leukemia (ALL), chronic lymphocytic leukemia (CLL), gliomas, and retinoblastomas.

### Effect of the Invention

In the present study, we examined the potential of micelle-based DNA vaccine platform comprising of TAA (SART3 or YB-1), CD40L and GM-CSF genes in mouse tumor models. Intraperitoneal administration of micelles with these genes prolonged the survival for peritoneal disseminated mice, and inhibited the growth and metastasis of subcutaneous tumors, where CTL/NK activities and the infiltration of CD4- and CD8a-positive lymphocytes (CTL) into tumor tissues were enhanced. These results suggest that the TAA/CD40L/GM-CSF-loading micelle is a highly potent DNA vaccine platform.

### BRIEF DESCRIPTION OF DRAWINGS

FIG. 1(A) A microscopic photograph showing the localization of polyplex micelles in spleen (left panel) and lymph nodes (center panel), and showing the co-localization of polyplex micelles and dendritic cells in lymph nodes (right panel). (B) A graph showing mGM-CSF expression.

FIG. 2(A) The scheme showing the vaccination schedule with polyplex micelle encapsulating therapeutic genes in CT26 peritoneal dissemination model. (B) The Kaplan-Meier survival curve demonstrating that the DNA vaccine encapsulating SART3, CD40L and GM-CSF significantly elongated the survival for mouse cancer models. (C) The scheme showing the vaccination schedule with the polyplex micelle. (D) Graphs showing the tumor weight of CT26 cancer and LLC subcutaneous tumors on day 14.

FIG. 3(A) Immunohistochemical images of lung tissues obtained from the mice with the indicated DNA vaccine or mock on day 28 after subcutaneous inoculation of LLC cancer. (B) Immunohistochemical images demonstrating the infiltration of CD4- and CD8a-positive T lymphocytes into the lung tissues.

FIG. 4(A) Graphs showing the NK activity (upper panel) and the CTL activity (lower panel). (B) Photographic images of tumor bearing mice. (C) A graph showing the CTL activity for long-term survivor mice received the DNA vaccine and for the control mice without the DNA vaccine. (D) The blocking experiments using ant-MHC class 1 (H-2L and -2D) antibodies or SART3 knockdown by siRNA transfection in CTL assay confirmed the specificity of CFSE-target cell killing to MHC and TAA species.

FIG. 5 Microscopic images of tissue sections from spleen, lymph nodes and tumors immunostained with the indicated antibodies and graphs showing the digitalized protein signals (red color in right panel) (left panel).

FIG. 6(A) Liposome-based DNA vaccine encapsulating SART3, CD40L and GM-CSF prolongs the survival for mice harboring CT26 peritoneal dissemination. (B) Subcutaneous administration of DNA vaccine in the groin region prolongs the survival for mice with peritoneal dissemination.

FIG. 7 CT26 colon cancer cells were implanted into the peritoneal cavity of BALB/c mice. One week later, a polyplex micelle with mouse MUC1/CD40L/GM-CSF genes was intraperitoneally administered, and then the survival of mice was monitored.

FIG. 8 CT26 colon cancer cells were implanted into the peritoneal cavity of BALB/c mice. One week later, a polyplex micelle with mouse survivine/CD40L/GM-CSF genes was intraperitoneally administered, and then the survival of mice was monitored.

FIG. 9 CT26 colon cancer cells were subcutaneously implanted in flank region, and one day later a block/homo mixed polyplex micelle encapsulating with SART3 plus indicated adjuvant genes (60 ug of pDNA, NP ratio=10) was administered into the peritoneal cavity of mice: (A) adjuvants=CD40L+GM-CSF; and (B) adjuvant=“28=scFv28-CD86 chimera”.

### MODE FOR CARRYING OUT THE INVENTION

Hereinafter, the present invention is described in detail. The embodiments described below are intended to be presented by way of example merely to describe the invention but not limited only to the following embodiments. The present invention may be implemented in various ways without departing from the gist of the invention.

All of the publications, published patent applications, patents and other patent documents cited in this application are herein incorporated by reference in their entirety. This application hereby incorporates by reference the contents of the specification and drawings in the Japanese Patent Application (No. 2013-079854) filed on Apr. 5, 2013, from which the priority was claimed.

In a first embodiment, the present invention provides a pharmaceutical composition for treating a tumor, which is a micelle encapsulating at least one tumor-associated antigen gene and at least one adjuvant gene. Hereinafter, the micelle may also be referred to as “DNA vaccine” of the present invention.

In the present invention, the tumor-associated antigen gene is at least one selected from the group consisting of squamous cell carcinoma antigen recognized by T cells 3 (SART3), Y-box binding protein 1 (YB-1), Mucin 1, cell surface associated (MUC1) and Survivin

The illustrative nucleotide sequences of the above listed TAA genes are summarized in the following Table 1. However, the nucleotide sequences of the TAA genes are not limited to those shown in the table, but also include nucleotide sequences of homologous genes thereof.

TABLE 1

| Gene | NCBI | | SEQ ID No. | SEQ ID No. |
|---|---|---|---|---|
| Name | Accession No. | Species | (gene) | (protein) |
| SART3 | NM_016926.1 | Mus musculus | 1 | 2 |
| YB-1 | NM_004559.3 | Homo sapiens | 3 | 4 |
| MUC1 | NM_013605.2 | Mus musculus | 5 | 5 |
| Survivin | AF077349.1 | Mus musculus | 7 | 8 |

Further, the adjuvant gene is at least one selected from the group consisting of Granulocyte-macrophage colony-stimulating factor (GM-CSF) and CD40L.

The illustrative nucleotide sequences of the above listed adjuvant genes are summarized in the following Table 2. However, the nucleotide sequences of the adjuvant genes are not limited to those shown in the table, but also include nucleotide sequences of homologous genes thereof.

TABLE 2

| Gene | NCBI | | SEQ ID No. | SEQ ID No. |
|---|---|---|---|---|
| Name | Accession No. | Species | (gene) | (protein) |
| GM-CSF | NM_009969.4 | Mus musculus | 9 | 10 |
| CD40L | NM_011616.2 | Mus musculus | 11 | 12 |

Alternatively, the adjuvant gene may be 28scFv(LH)-CD86 chimera or variants thereof, which have an activity of 28scFv(LH)-CD86 chimera. The polynucleotides including 28scFv(LH)-CD86 chimera or variants thereof may be selected from the group consisting of (a) to (e) below:

(a) a polynucleotide comprising the nucleotide sequence of SEQ ID NO: 13;

(b) a polynucleotide encoding a protein consisting of the amino acid sequence of SEQ ID NO: 14

(c) a polynucleotide encoding a protein consisting of an amino acid sequence wherein 1 to 40 amino acids are deleted, substituted, inserted and/or added in the amino acid sequence of SEQ ID NO: 14, and having an activity of 28scFv(LH)-CD86 chimera;

(d) a polynucleotide encoding a protein having an amino acid sequence having at least 85% homology to the amino acid sequence of SEQ ID NO: 14, and having an activity of 28scFv(LH)-CD86 chimera; and,

(e) a polynucleotide which hybridizes to a polynucleotide consisting of a nucleotide sequence complementary to the nucleotide sequence of SEQ ID NO: 13 under stringent conditions, and which encodes a protein having an activity of 28scFv(LH)-CD86 chimera.

The polynucleotides including 28scFv(LH)-CD86 chimera or variants thereof may be used in combination with any one or both of GM-CSF and CD40L.

As used herein, the term “polynucleotide” means a DNA or RNA.

As used herein, the term “polynucleotide which hybridizes under stringent conditions” refers to a polynucleotide obtained by a colony hybridization method, a plaque hybridization method, a Southern hybridization method or the like, using as a probe, for example, a polynucleotide consisting of a nucleotide sequence complementary to the nucleotide sequence of SEQ ID NO: 13, or the whole or part of a polynucleotide consisting of the nucleotide sequence encoding the amino acid sequence of SEQ ID NO: 14. For the methods of hybridization, there are used the methods described in, e.g., “Sambrook & Russell, Molecular Cloning; A Laboratory Manual Vol. 3, Cold Spring Harbor, Laboratory Press 2001” and “Ausubel, Current Protocols in Molecular Biology, John Wiley & Sons 1987-1997”, etc.

As used herein, the term “stringent conditions” may be any of low stringent conditions, moderate stringent conditions or high stringent conditions. The term “low stringent conditions” are, for example, 5×SSC, 5× Denhardt's solution, 0.5% SDS, 50% formamide at 32° C. The term “moderate stringent conditions” are, for example, 5×SSC, 5× Denhardt's solution, 0.5% SDS, 50% formamide at 42° C., or 5×SSC, 1% SDS, 50 mM Tris-HCl (pH 7.5), 50% formamide at 42° C. The term “high stringent conditions” are, for example, 5×SSC, 5× Denhardt's solution, 0.5% SDS, 50% formamide at 50° C. or 0.2×SSC, 0.1% SDS at 65° C. Under these conditions, a DNA with higher homology is expected to be obtained efficiently at higher temperatures, although multiple factors are involved in hybridization stringency including temperature, probe concentration, probe length, ionic strength, time, salt concentration and others, and one skilled in the art may appropriately select these factors to achieve similar stringency.

When commercially available kits are used for hybridization, for example, an Alkphos Direct Labeling and Detection System (GE Healthcare) may be used. In this case, according to the attached protocol, after cultivation with a labeled probe overnight, the membrane is washed with a primary wash buffer containing 0.1% (w/v) SDS at 55° C., thereby detecting hybridized DNA. Alternatively, in producing a probe based on the nucleotide sequence complementary to the nucleotide sequence of SEQ ID NO: 13 or on the entire or part of the nucleotide sequence encoding the amino acid sequence of SEQ ID NO: 14, hybridization can be detected with a DIG Nucleic Acid Detection Kit (Roche Diagnostics) when the probe is labeled with digoxygenin (DIG) using a commercially available reagent (e.g., a PCR Labeling Mix (Roche Diagnostics), etc.).

In addition to those described above, other polynucleotides that can be hybridized include DNAs having 70% or higher, 71% or higher, 72% or higher, 73% or higher, 74% or higher, 75% or higher, 76% or higher, 77% or higher, 78% or higher, 79% or higher, 80% or higher, 81% or higher, 82% or higher, 83% or higher, 84% or higher, 85% or higher, 86% or higher, 87% or higher, 88% or higher, 89% or higher, 90% or higher, 91% or higher, 92% or higher, 93% or higher, 94% or higher, 95% or higher, 96% or higher, 97% or higher, 98% or higher, 99% or higher, 99.1% or higher, 99.2% or higher, 99.3% or higher, 99.4% or higher, 99.5% or higher, 99.6% or higher, 99.7% or higher, 99.8% or higher or 99.9% or higher identify with to the DNA of SEQ ID NO: 13, or the DNA encoding the amino acid sequence of SEQ ID NO: 14, as calculated by homology search software, such as FASTA and BLAST using default parameters.

Identity between amino acid sequences or nucleotide sequences may be determined using algorithm BLAST by Karlin and Altschul (Proc. Natl. Acad. Sci. USA, 87: 2264-2268, 1990; Proc. Nail Acad. Sci. USA, 90: 5873, 1993). Programs called BLASTN, BLASTX, BLASTP, tBLASTN and tBLASTX based on the BLAST algorithm have been developed (Altschul S. F. et al., J. Mol. Biol. 215: 403, 1990). When a nucleotide sequence is sequenced using BLASTN, the parameters are, for example, score=100 and wordlength=12. When an amino acid sequence is sequenced using BLASTP, the parameters are, for example, score=50 and wordlength=3. When BLAST and Gapped BLAST programs are used, default parameters for each of the programs are employed.

The polynucleotides of the present invention described above can be acquired by known genetic engineering techniques, known methods for synthesis, and so on.

Examples of tumor include (1) sarcomas such as osteosarcoma and soft tissue sarcoma, (2) carcinomas such as carcinoma of the breast, carcinoma of the lung, carcinoma of the bladder, carcinoma of the thyroid gland, carcinoma of the prostate, carcinoma of the colon, colorectal carcinoma, carcinoma of the pancreas, carcinoma of the stomach, carcinoma of the liver, carcinoma of the uterus, carcinoma of the cervix and carcinoma of the ovary, (3) lymphomas such as Hodgkin lymphoma and non-Hodgkin lymphoma, (4) neuroblastomas, (5) melanomas, (6) myelomas, (7) Wilms tumors, (8) leukemias such as acute myelocytic leukemia (AML), chronic myelocytic leukemia (CML), acute lymphocytic leukemia (ALL) and chronic lymphocytic leukemia (CLL), (9) gliomas, and (10) retinoblastomas.

The tumor-associated antigen (TAA) gene and adjuvant gene may be inserted into a suitable expression cassette(s) in the form of an expression vector. A suitable expression cassette at least contains the following constituents (i) to (iii):

(i) promoter capable of transcribing in target tumor cells;

(ii) gene ligated in-frame to the promoter; and

(iii) sequence encoding transcription termination and polyadenylation signal of RNA molecule.

Examples of promoters capable of transcribing in target tumor cells include, but are not limited to, CMV, CAG, LTR, EF-1α and SV40 promoters.

Examples of the expression cassette is not limited as long as it can express the inserted gene and include pEGFP-C1™ (Clontech), pCMV-HA™ (Clontech), pMSCVpuro™ (Clontech), pEF-DEST51™ (Invitrogen), pCEP4™ (Invitrogen), ViraPower II Lentiviral Gateway System™ (Invitrogen), pVIVO1-mcs2 plasmid (Invitrogen).

In a case where the composition of the present invention is used as a DNA vaccine, gene transfer may be accomplished either by direct administration in which the micelle is directly injected into the body or by indirect administration in which the vector is infected into subject's own cells or other cells for gene transfer, and the infected cells are then injected into a target site. For direct injection of the vector, intraperitoneal injection or the like may be used.

Alternatively, the micelle of the present invention may be a polyion complex micelle including polyplex micelles or liposomes. Using such micelles, the TAA gene and the adjuvant genes encapsulated therein are introduced into a cell by lipofection. Then, the resulting cells are administered systemically, for example, by the intravenous or intraarterial route. They may be administered locally to a target tissue, e.g., brain, etc.

Examples of lipids which may be used to form the polyion complex micelle include phospholipids, cholesterols and nitrogen-containing lipids. Commonly preferred are phospholipids, including natural phospholipids such as phosphatidylcholine, phosphatidylserine, phosphatidylglycerol, phosphatidylinositol, phosphatidylethanolamine, phosphatidic acid, cardiolipin, sphingomyelin, egg yolk lecithin, soybean lecithin, and lysolecithin, as well as hydrogenated products thereof obtained in a standard manner. It is also possible to use synthetic phospholipids such as dicetyl phosphate, distearoylphosphatidylcholine, dipalmitoylphosphatidylcholine, dipalmitoylphosphatidylethanolamine, dipalmitoylphosphatidylserine, eleostearoylphosphatidylcholine, eleostearoylphosphatidylethanolamine as well as homo-poly{N′—[N-(2-aminoethyl)-2-aminoethyl]aspartamide} P[Asp(DET)] and block-catiomer poly(ethyleneglycol) (PEG)-b-P[Asp(DET)].

The preparation of micelle is not limited in any way as long as the resulting micelles hold DNAs. The micelles may be prepared in a conventional manner, for example, by reversed-phase evaporation, ether injection, surfactant-based techniques, etc.

Lipids including these phospholipids may be used either alone or in combination. Since DNA molecules are electrically negative, the binding rate between the DNA, i.e., the TAA and adjuvant genes, and the micelles may be enhanced by using a lipid containing an atomic group(s) having a cationic group (e.g., ethanolamine or choline). In addition to these phospholipids, it is also possible to use other additives such as cholesterols, stearyl amine, α-tocopherol and the like in the micelle, which are generally known as micelle-forming additives. The micelles thus obtained may further comprise a membrane fusion promoter (e.g., polyethylene glycol) in order to enhance their uptake into cells at the affected area or of the target tissue.

The DNA vaccine or pharmaceutical composition according to the present invention may be formulated in a routine manner and may comprise pharmaceutically acceptable carriers to suspend the micelles. Such carriers may be additives and include water, buffers such as phosphate buffer saline, pharmaceutically acceptable organic solvents, collagen, polyvinyl alcohol, polyvinylpyrrolidone, carboxyvinyl polymers, carboxymethylcellulose sodium, sodium polyacrylate, sodium alginate, water-soluble dextran, carboxymethyl starch sodium, pectin, methylcellulose, ethylcellulose, xanthan gum, gum arabic, casein, agar, polyethylene glycol, diglycerine, glycerine, propylene glycol, petrolatum, paraffin, stearyl alcohol, stearic acid, human serum albumin, mannitol, sorbitol, lactose, and surfactants acceptable as pharmaceutical additives, etc.

The above additives may be selected alone or in combination from among those listed above, depending on the dosage form of each therapeutic agent of the present invention. For example, for use as injectable formulations, the purified vector may be dissolved in a solvent (e.g., physiological saline, buffer, glucose solution) and then supplemented with Tween 80, Tween 20, gelatin, human serum albumin or the like. Alternatively, the ingredients may be lyophilized for use as dosage forms that are reconstituted before use. Examples of excipients for lyophilization include sugars such as mannitol, glucose, lactose, sucrose, mannitol and sorbitol; starches such as those derived from corn, wheat, rice, potato and other plants; celluloses such as methylcellulose, hydroxypropylmethylcellulose and carboxymethylcellulose sodium; gums such as gum arabic and gum tragacanth; as well as gelatin, collagen and so on.

In a second embodiment, the present invention provides a method for preventing and/or treating a tumor, comprising administering a micelle encapsulating at least one tumor-associated antigen gene and at least one adjuvant gene to a subject in need of such treatment.

The subject to be administered with the DNA vaccine of the present invention include, for example, humans and all other mammals such as non-human primates (e.g., monkeys), rodents (e.g., mice and rats), rabbits, goats, sheep, pigs, cattle and dogs, with humans being more preferred. The subject may also be, for example, those suffering from cancer such as colon cancer or those suspected to have cancer such as colon cancer.

The dosage of the DNA vaccine of the present invention will vary depending on the age, sex and symptoms of a subject, the route of administration, the frequency of administration, and the intended dosage form. The mode of administration is selected as appropriate for the age and symptoms of a subject. The effective dosage of the DNA vaccine is an amount of the vaccine required to reduce the signs or condition of the disease. The therapeutic effect and toxicity of such a DNA vaccine may be determined by standard pharmaceutical procedures in cell culture or in laboratory animals, for example, by ED50 (therapeutically effective dose in 50% of the population) or LD50 (lethal dose for 50% of the population) assay.

The route of administration may be selected as appropriate and examples include, but are not limited to, percutaneous, intranasal, transbronchial, intramuscular, intraperitoneal, intravenous and subcutaneous routes. Particularly preferred routes are intraperitoneal administration, subcutaneous administration and so on. Inoculation may be made at a single site or at multiple sites.

The kind of expression vector may be selected as appropriate and examples include, but are not limited, to a plasmid vector. Commonly preferred vectors, such as adeno, adeno-associated, vaccinia, Sendai and pox viral gene vectors, are also possible to use as for the present invention. The dose ratio between therapeutic and toxic effects is a therapeutic index and can be expressed as ED50/LD50. In humans, the single dosage of the vaccine of the present invention is about 1 μg to 1000 μg, preferably about 10 to 500 μg, more preferably about 50 to 250 μg. The frequency of administration may be once or more as long as side effects are within a clinically acceptable range.

### EXAMPLES

The present invention is now described in detail by way of using working examples below. However, the scope of the present invention shall not be limited to the examples but should be appreciated by the scope of the claims attached.

### Materials and Methods

### Plasmid DNA Construction

Expression plasmids of GM-CSF, CD40L, squamous cell carcinoma antigen recognized by T cells 3 (SART3) and Y-box binding protein 1 (YB-1) genes were constructed as follows; The open-reading frame of mouse GM-CSF, CD40L, SART3 or partial sequences of human YB-1 genes (corresponding to 1-121 amino acids) was integrated at the multi-cloning sites in the pVIVO1-mcs2 plasmid (Invivogen). The plasmid DNA was amplified in Escherichia coli DH5A competent cells and purified using EndoFree Plasmid Giga Kit (QIAGEN inc.).

Preparation of Polyplex Micelles Encapsulating pDNA

Homo-poly{N′—[N-(2-aminoethyl)-2-aminoethyl]aspartamide} P[Asp(DET)] (degree of polymerization (DP): 55) and block-catiomer poly(ethyleneglycol) (PEG)-b-P[Asp(DET)] (Mw of PEG: 12000; DP: 65), synthesized as previously reported [ref 19, 22], were kindly provided from NOF corp. (Kawasaki, Japan). Polyplex micelles were prepared by mixing pDNA (50 μg), PEG-b-P[Asp(DET)] and P[Asp(DET)] in 10 mM HEPES buffer (pH 7.3) at the block/homo ratio of 7/3 and the N/P ratio of 10 (N=total amines in polycations; P=total phosphate anions in pDNA). Dynamic light scattering (DLS) measurement was carried out at 25° C. using an ELSZ-SV2 (Otsuka Electronics Co., Ltd.), equipped with a detection angle 160° of a He—Ne ion laser (633 nm) as the incident beam. The rate of decay in the photon correlation function was analyzed by the cumulant method, and the corresponding hydrodynamic diameter of the polyplexes was then calculated by the Stokes-Einstein equation.

### Cell Lines

Murine colorectal carcinoma (CT26), lymphoma (YAC-1) and Lewis lung carcinoma (3LL/LLC) were obtained from the American Type Culture Collection. These cells were maintained in RPMI1640 medium (Nacalai tesque, Ltd.) supplemented with 10% heat-inactivated fetal bovine serum (FBS, Wako Pure Chemical Industries, Ltd.), 100 U/ml penicillin and 100 μg/ml streptomycin at 37° C. in humidified incubators containing 5% CO₂.

### Animals

BALB/c AnNCrlCrlj mice (female, 6 weeks old) and C57BL/6J (female, 6 weeks old) were purchased from Charles River Laboratories (Yokohama, Japan). Animals were housed in a temperature-controlled room under 12/12 hours light/dark cycles and accessed the intake of food and water ad libitum. All animal procedures were approved and carried out in accordance with the institutional Guidelines for Animal Experiments from the Animal Care and Use Committee at Kyushu University.

Polyplex Micelle Distribution After i.p. Administration

PEG-b-P[Asp(DET)] was labeled with Fluolid fluorescence, as previously demonstrated [Kumagai A]. Fluorescence-labeled PEG-b-P[Asp(DET)]/P[Asp(DET)] mixed micelles with pVIVO-1-mock were administered into the peritoneal cavity of mice. At 24 hours later, several organ tissues (liver, spleen and lymph nodes) were obtained, and the tissue localization of fluorescence-labeled polyplex micelles was examined under laser confocal microscope.

Localization of Gene Expression from Polyplex Micelle After i.p. Administration

PEG-b-P[Asp(DET)]/P[Asp(DET)] mixed micelles encapsulating GM-CSF gene were administered into the peritoneal cavity of mice, and the organ tissues (liver, spleen, lung, kidney and lymph node) were obtained at day 1, 3 and 7 (n=4 in each). Total RNA samples were extracted using RNA extraction kit (Roche), after which the synthesized cDNA samples were subjected to real-time RT-PCR analysis for GM-CSF gene expression, as previously reported [Ohgitani M].

### Mouse Tumor Model and Vaccination Protocols

Vaccination protocol was designed as a therapeutic vaccine for adjuvant settings to mimic cancer subjects with micro-metastasis after surgical resection. We prepared two types of syngeneic tumor models of peritoneal dissemination and subcutaneous tumors that were developed with murine colorectal cancer CT26 cells in BALB/c mice and murine lung cancer LLC cells which has high metastatic potentials in C57/BL6 mice.

For peritoneal dissemination model, CT26 cells (1×10⁵ cells/mouse) were inoculated into the peritoneal cavity of BALB/c mice (day 0). Thereafter, polyplex micelles encapsulating with the indicated genes (Table 3) were intraperitoneally administered four times at every one-week interval (day 1, 8, 15 and 22). The survival of the mice was monitored until day 80 after the first inoculation of CT26 cells to evaluate the anti-tumor efficacy of polyplex micelle-encapsulating DNA vaccine. To examine the acquirement of CT26-specific rejection immunity, mice survived more than 80 days (long-term survivor) were subcutaneously inoculated with CT26 cells (1×10⁶ cells/mouse) at the flank region (re-challenge experiment). The occurrence and growth of subcutaneous tumor at injected site was carefully observed for 60 days after the re-challenge of CT26 cells. In subsets of experiments, splenocyte cells were freshly isolated from long-term survivor mice and subjected to the CTL and NK cytotoxic assays to explore the acquirement of cellular anti-tumor immunity.

For subcutaneous tumor model, syngeneic CT26 cells or LLC cells (both 1×10⁶ cells/mouse) were subcutaneously inoculated at the flank region of BALB/c or C57/BL6 mice, respectively (day 0). Then, polyplex micelles encapsulating with the indicated genes (Table 3) were intraperitoneally administered four times at every one-week interval (day 1, 8, 15 and 22). Mice were sacrificed on day 14 for BALB/c mice and on day28 for C57/BL6 mice except for the mice died for less than 28 days. The weight of subcutaneous tumors was compared between the groups to evaluate the anti-tumor effect of polyplex micelle-carried DNA vaccines. Tumor and several organ tissues were obtained and snap-frozen in OCT compounds with liquid nitrogen for histological analysis to examine the presence of lung metastasis in 3LL/LLC tumor models and for immunohistochemistry of immune cells infiltration in spleen, lymph nodes and tumor tissues. In subsets of experiments, splenocyte cells were freshly isolated and co-cultured with the target CT26, LLC, or YAC-1 cells for CTL and NK cytotoxic assays.

### Subcutaneous Administration of DNA Vaccine in the Groin Region

pDNAs of SART3, CD40L and GM-CSF (total 50 ug) were encapsulated with PEG-b-[Pasp(DET)]/Pasp(DET) at 10 of N/P ratio. The polyplex micelle-based DNA vaccine was subcutaneously administered in the groin region of mice harboring CT26 peritoneal dissemination.

### CTL and NK Assay (CFSE-Based Cytotoxicity Assay)

CT26 or LLC cells were treated with 20 Gy irradiation for arrest of cell growth. Splenocyte (5×10⁷ cells) isolated from mice harboring CT26 and LLC subcutaneous tumors were co-incubated with irradiated CT26 or LLC/3LL (5×10⁶ cells) in 20 ml of RPMI-1640 medium (Nacalai tesque, Ltd.) supplemented with 10% FBS, 5×10⁻⁵M 2-mercaptoethanol, 100 U/ml penicillin and 100 μg/ml streptomycin at 37° C. in humidified incubators containing 5% CO₂. After 72 hr incubation, these splenocyte cells were harvested and used as effector cells for the CTL and NK assays, as previously described [ref 23].

Target cells of CT26 or 3LL/LLC for CTL assays and YAC-1 for NK assays were resuspended with the RPMI-1640 medium at the density of 20×10⁶ cells/mL and labeled with 10 μM of CFSE (Dojindo) for 10 minutes at 37° C. The reaction was stopped by the addition of an equal volume of fetal calf serum (FCS). After washing with RPMI medium twice, the CFSE-labeled target cells were immediately mixed with the effector cells at different target/effector (T/E) ratios of 1/0, 1/25, 1/50 or 1/100 (T: 1×10⁴ cells/E: 0, 25×10⁴, 50×10⁴, 100×10⁴ cells, respectively) in 200 μl of the RPMI medium, and incubated in a humidified atmosphere of 5% CO₂ and 37° C. for another 6 hours. Flow-Count Fluorospheres (10,000 in each sample, Coulter Corporation) and propidium iodide (1 μg/mL, a marker of dead cells) were added to the cell mixture just prior to the analysis of flow cytometry (BD FACS CANT-II). For facilitating the number of target cells, 2,000 microbeads was referred to event count on Cell Quest software. The percentage of survival was calculated as follows: [number of viable CFSE⁺ target cells for T/E ratio 1/25-1/100] divided by [that for T/E ratio 1/0]×100.

### MHC and SART3-Blocking Experiments in CTL Assay

To analyze the major histocompatibility complex (MHC) restriction of the target cell lysis in CTL assay, blocking studies were performed using neutrizing antibodies. Target cells were incubated with saturated concentrations of anti-MHC class I monoclonal antibodies (H-2L^{d}: 28-14-8, BioLegend and H-2K^{d}: SF1-1.1.1, eBioScience) for 30 minutes before mixing with effecter cells. Alternatively, to confirm the TAA specificity of the target cell lysis in CTL assay, SART3 expression was knock-downed in CT26 by siRNA (sense: 5′-CUACAGUCAGUACCUAGAUTT-3′ (SEQ ID NO: 15) and antisense: 5′-AUCUAGGUACUGACUGUAGTT-3′ (SEQ ID NO: 16) using lipofectamine 2000 in accordance with the manufacturer's protocol (Life techonology™). The efficiency of knocking down mRNA was confirmed by real-time RT-PCR methods. After the blocking MHC molecules or knocking down SART3 expression, the treated CT26 cells were mixed with effecter cells at several E/T ratios for CTL assay.

### Real-Time RT-PCR

Total RNA was extracted using illustra™ RNAspin Mini RNA Isolation Kit (GE Healthcare) and the cDNA was synthesized using Transcriptior First Strand cDNA synthesis Kit (Roche Applied Science). The real-time RT-PCRs for mouse GM-CSF, SART3 and β-actin (housekeeping gene) were performed using the published primer sets for GM-CSF and beta-actin, and 5′-GTGAGCTCTTCCCCCTGAC-3′ (SEQ ID NO: 17) and 5′-CATGCTGATCTCATCGTGGA-3′ (SEQ ID NO: 18) for SART3 in the LightCycler480 II system (Roche Diagnostics), as previously reported [Ref 24 ].

### Liposome-Based DNA Encapsulating SART3, CD40L and GM-CSF

pDNAs of SART3, CD40L and GM-CSF (total 50 ug) were encapsulated with liposome (Coatsome EL-01-C, NOF corp.) in accordance with the manufacture's protocol. The liposome-based DNA vaccine was intraperitoneally administered in mice harboring CT26 peritoneal dissemination, as similarly as the polyplex micelle-based DNA vaccine.

### Immunohistochemistry

Tumor, lung and the immune organ tissues (spleen, liver and lymph nodes) in subcutaneous tumor models were sectioned in 10 μm thickness and fixed ice-cold Acetone for 10 minutes. The sections were immersed with 3% H₂O₂ and 1% bovine serum albumin to block the endogenous peroxidase activity. The specimens were incubated with a primary antibody for CD4 (1:250. #100505, BioLegend), CD8a (1:1000, #100701, BioLegend), CD11c (1:500, ab33483, Abcam), or GM-CSF (1:1000, ab13789, Abcam) at room temperature for one hour and then with the VECTASTAIN biotin/avidin system (Vector, USA), followed by the visualization with 3,3′-diaminobenzidine (DAB) and hematoxylin-counterstain. The signal of immunostaining was taken as digital image data under optical microscope (ECLIPSE 55i, Nikon) and quantitated the expression level using NIS-Elements D 3.2 quantitative analysis program (NIKON).

### Statistical Analysis

Results are represented as means±standard deviation (SD). The differences were statistically analyzed using Student's t-test between two groups or analysis of variance (ANOVA) between multiple groups. Survival curve was evaluated by Kaplan-Meier method and analyzed with a log-lank test. P values less than 0.05 were considered statistically significant.

### Validation of MUC1 and Survivine as TAA for Gene Vaccine

CT26 colon cancer cells were implanted into the peritoneal cavity of BALB/c mice. One week later, polyplex micelles with mouse MUC1/CD40L/GM-CSF or mouse survivin/CD40L/GM-CSF genes were intraperitoneally administered, and then the survival of mice was monitored.

### Chimera of Single Chain of Variable Fragment of Anti-CD28 Antibody Fused to CD86 Molecule has an Adjuvant Effect

The sequence of single chain of variant fragment against CD28, a co-stimulatory molecule (scFv28: 28^{th} to 140^{th} and 156^{th} to 278^{th} amino acid residues of SEQ ID NO: 14), was collected from the information of antagonistic anti-CD28 antibody's sequence, as previously reported by Kumagai and colleagues. Then, we generated the chimera adjuvant gene: scFv28-CD86 (SEQ ID NO: 13), which was scFv28 sequence fused to just after signal sequence of CD86 gene (signal sequence of CD86: 1^{st} to 27^{th} and 284^{th} to 499^{th} amino acid residues of SEQ ID NO: 14) via two spacer sequences (1^{st} spacer sequence: 141^{st} to 155^{th} amino acid residues of SEQ ID NO: 14, 2^{nd} spacer sequence: 279^{th} to 283^{rd} amino acid residues of SEQ ID NO: 14). CT26 colon cancer cells (1×10⁶/mouse) were subcutaneously implanted at flank region, and one day later, SART3 plus indicated adjuvant genes-loading DNA vaccines (60 ug of pDNA, NP ratio=10) were administered into the peritoneal cavity of the mice. After the repeated vaccinations (4 times with one week interval), subcutaneous tumors were obtained at day 28 and compared the tumor weight between the DNA vaccine and mock groups.

### REFERENCES

22. Itaka K, Ishii T, Hasegawa Y, Kataoka K (2010) Biodegradable polyamino acid-based polycations as safe and effective gene carrier minimizing cumulative toxicity. Biomaterials 31: 3707-3714.
23. Furugaki K, Pokorna K, Le Pogam C, Aoki M, Reboul M, et al. (2010) DNA vaccination with all-trans retinoic acid treatment induces long-term survival and elicits specific immune responses requiring CD4+ and CD8+ T-cell activation in an acute promyelocytic leukemia mouse model. Blood 115: 653-656.
24. Ohgidani M, Furugaki K, Shinkai K, Kunisawa Y, Itaka K, et al. (2013) Block/homo polyplex micelle-based GM-CSF gene therapy via intraperitoneal administration elicits antitumor immunity against peritoneal dissemination and exhibits safety potentials in mice and cynomolgus monkeys. J Control Release 167: 238-247.

### Results

### Polyplex Micelle Characterization

The polyplexes mixed PEG-P[Asp(DET)], P[Asp(DET)] and pDNA (50 μg) (block/homo=7/3, NP=10) formed the micelles in diameter at 91.3±3.16 nm. The polyplex micelles showed neutral ζ-potential value 1.55±1.16 (mV).

### Polyplex Micelle Tissue Localization and Gene Expression

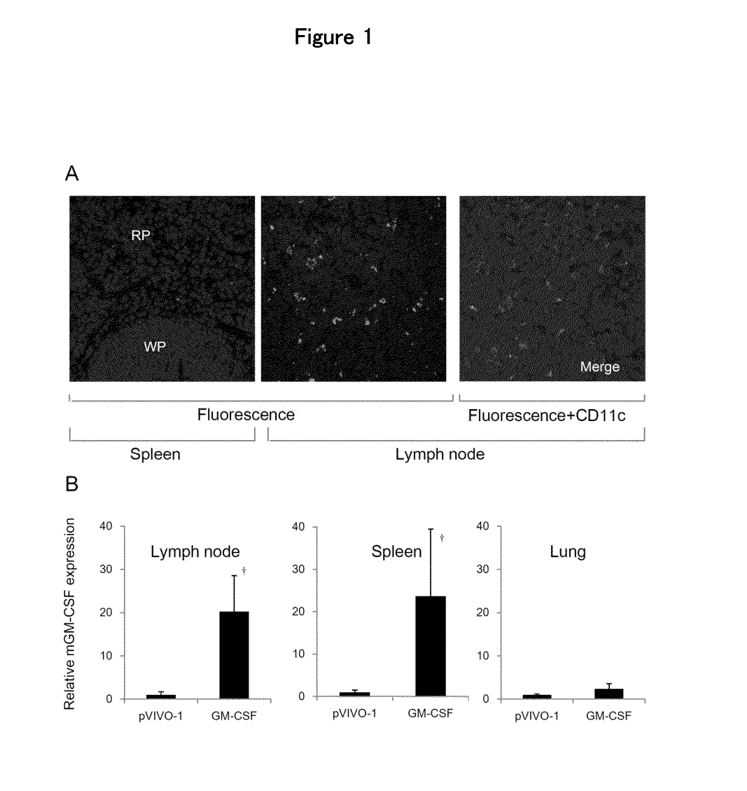

The polyplexes mixed PEG-P[Asp(DET)] with fluorescence, P[Asp(DET)] and pDNA (50 μg) (block/homo=7/3, NP=10) formed the micelles were mainly localized in spleen and lymph nodes (FIG. 1A). We examined the expression level and distribution of therapeutic gene: GM-CSF by the qRT-PCR in various normal organ tissues on day 1, 3, 7 after i.p. administration of GM-CSF pDNA carried-polyplex micelles. The polyplex micelles induced 20-fold higher expression of GM-CSF in lymph node and 24-fold higher expression in spleen (FIG. 1B) compared with mock group. On the other hand, no significant increase was detected in lung (FIG. 1B), liver, and kidney.

Polyplex Micelle-Based DNA Vaccine with SART3, CD40L and GM-CSF Genes Prolongs the Survival for Mice Harboring Peritoneal Dissemination

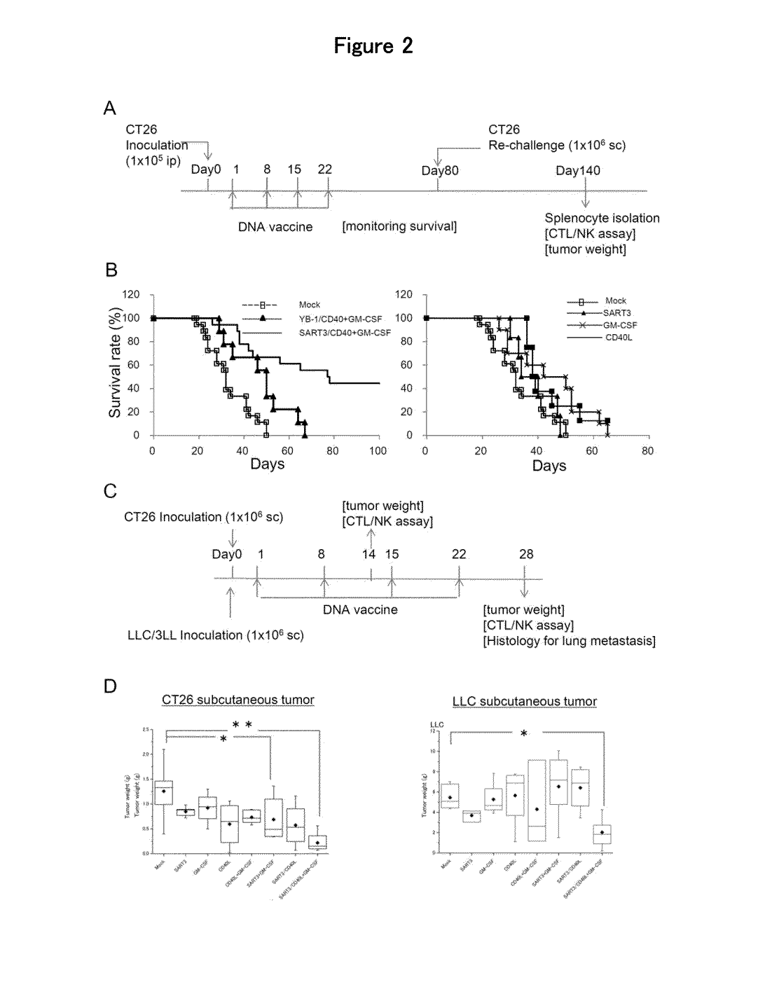

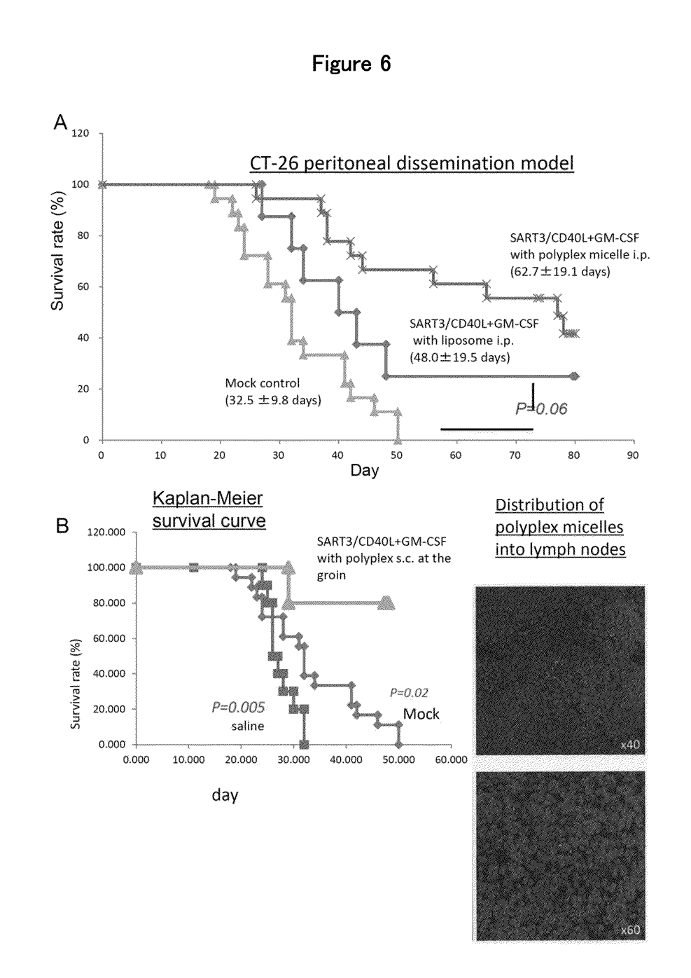

We compared the survival periods for mice harboring peritoneal dissemination of CT26 cancer between each group as indicated in Table 3. The polyplex micelles encapsulating SART3 alone (38.7±6.9 days), CD40L alone (44.0±9.9 days) or GM-CSF alone (44.3±13.3 days) did not prolong the survival compared with the mock control (32.5±9.8 days). Moreover, the combination of CD40L+GM-CSF (39.1±10.3 days), SART3+CD40L (36.0±9.1 days) or SART3+GM-CSF (50.3±9.8 days) had no significant or much less elongation for the survival compared with the mock control. The polyplex micelles with three combination of TAA: SART3, CD40L and GM-CSF only achieved the significantly longer survival (62.7±19.1 days) compared with mock (32.5±9.8 days) (FIG. 6A). The Kaplan-Meier analysis shows a significant increase in survival rate for the DNA vaccine with SART3 or YB-1, CD40L and GM-CSF combinations than the mock control (P=0.00003; FIG. 2B left panel). To the contrary, the survival rates were not improved by the polyplex micelles with either single gene (FIG. 2B right panel) or naked plasmids (SART3/CD40L+GM-CSF) without the polyplex micelles (data not shown).

TABLE 3
Therapeutic genes encapsulated with polyplex micelles and
their median survival periods in CT26 peritoneal
dissemination model.

| | Survival days) |
|---|---|
| Mock (50 μg) | 32.5 ± 9.8 |
| SART3 (25 μg) + Mock (25 μg) | 38.7 ± 6.9 |
| CD40L (25 μg) + Mock (25 μg) | 44.0 ± 9.9 |
| GM-CSF (25 μg) + Mock (25 μg) | 44.3 ± 13.3 |
| SART3 (25 μg) + CD40L (25 μg) | 36.0 ± 9.1 |
| SART3 (25 μg) + GM-CSF (25 μg) | 50.3 ± 9.8* |
| CD40L (25 μg) + GM-CSF (25 μg) | 39.1 ± 10.3 |
| SART3/CD40L (25 μg) + GM-CSF (25 μg) | 62.7 ± 9.8** |

Values are represented as means ± SD for median survival (n = 6-19).
*P < 0.05,
**P < 0.0001 versus Mock control
SART3: squamous cell carcinoma antigen recognized by T cells 3, Polyplex micelle-based DNA vaccine with SART3, CD40L and GM-CSF genes inhibits the growth of subcutaneous tumors.

As shown in FIG. 2C, we also examined the inhibitory effect of DNA vaccine on the growth in subcutaneous CT26 or LLC/3LL tumor models. When monitoring the same CT26 tumors as peritoneal dissemination model, the DNA vaccine encapsulating SART3, CD40L and GM-CSF combination significantly decreased the tumor growth compared with the mock control (0.22±0.17 g versus 1.3±0.46 g; P=0.0001), while the less or not significant inhibition in tumor growth were observed in the treatment groups with CD40L (0.92±0.28 g; P=0.2), SART3 (0.89±0.09 g; P=0.06), GM-CSF (0.60±0.40 g; P=0.05), CD40L+GM-CSF (0.58±0.40 g; P=0.05), SART3+GM-CSF (0.73±0.12 g; P=0.02) and SART3+CD40L (0.69±0.49 g; P=0.045), as shown in FIG. 2D (left panel).

To validate the efficacy of the DNA vaccine for another MHC and tumor species, we examined the inhibitory effect on the growth of the subcutaneous tumor of LLC/3LL cells in CB57/BL6 mice which have a different haplotype of MHC class 1, H-2B. As shown in FIG. 2D (right panel), the growth of subcutaneous LLC tumor was significantly suppressed for the DNA vaccine with SART3, CD40L and GM-CSF (2.0±1.3 g) compared with mock (5.5±1.1 g; P=0.0004). In contrast, there were no significant differences for other treatment groups with SART3 (3.7±0.5 g), GM-CSF (5.3±1.5 g), CD40L (5.7±2.7 g), CD40L+GM-CSF (4.3±3.5 g), SART3+GM-CSF (6.5±3.1 g), or SART3+CD40L (6.4±2.0 g) compared with the mock control (FIG. 2D left panel).

Polyplex Micelle-Based DNA Vaccine with SART3, CD40L and GM-CSF Genes Inhibits the Lung Metastasis of LLC Subcutaneous Tumors.

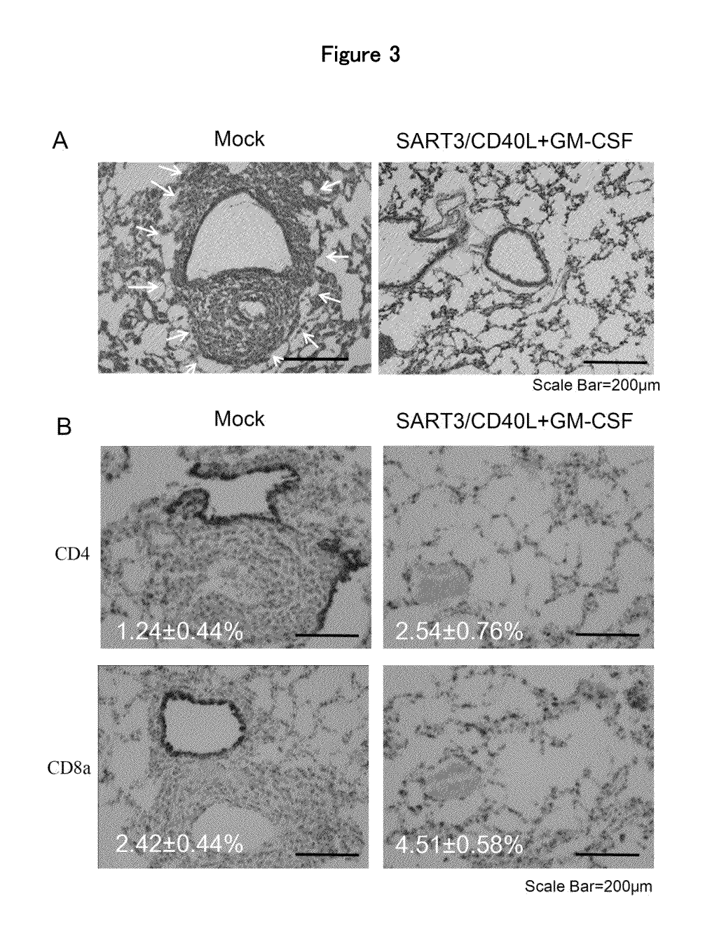

Since LLC/3LL cancer is known to exhibit a highly metastatic phenotype, we monitored the occurrence of lung metastasis in mice harboring subcutaneous LLC tumors for four weeks after i.p. administration of the polyplex micelles with the DNA vaccine or mock gene. As expected, histological examination depicted lung metastasis at 100% (4/4 cases) in the mock control (FIG. 3A, left panel). On the other hands, all mice administered the DNA vaccine with SART3, CD40 and GM-CSF combination developed no lung metastasis (0/4 cases; FIG. 3A, right panel) accompanied by greater regression in tumor growth (FIG. 2D, left panel). Instead of tumor metastatic nodules, many immune cells were present in lung tissues for the DNA vaccine group. Thus, we carried out the immunohistochemical analysis for GM-CSF, CD11c, CD4 and CD8a, and found that the infiltrations of GM-CSF, CD4 and CD8a-positive immune cells were increased with two-fold degree compared with the mock control (P=0.006, 0.024, and 0.001, n=4 in each, respectively; FIG. 3B).

Subcutaneous Administration of DNA Vaccine in the Groin Region Prolongs the Survival for Mice with Peritoneal Dissemination

CT26 Left panel demonstrates that the polyplex micelle-based DNA vaccine prolonged the survival compared with the mock and saline controls (P=0.02 and P=0.005, respectively, for log-rank test). Right panel shows that the Fluolid-labeled polyplex micelles were distributed into lymph nodes at the groin region in the mice. (FIG. 6B)

### Liposome-Based DNA Encapsulating SART3, CD40L and GM-CSF Prolongs the Survival for Mice Harboring CT26 Peritoneal Dissemination

CT26 The liposome-based DNA vaccine prolonged the survival (48.0±19.5 days) compared with the mock control (32.5±9.8 days; P=0.06 for log-rank test). (FIG. 6A)

CTL and NK Cytotoxicities are Enhanced by Polyplex Micelle-Based DNA Vaccine with SART3, CD40L and GM-CSF Genes

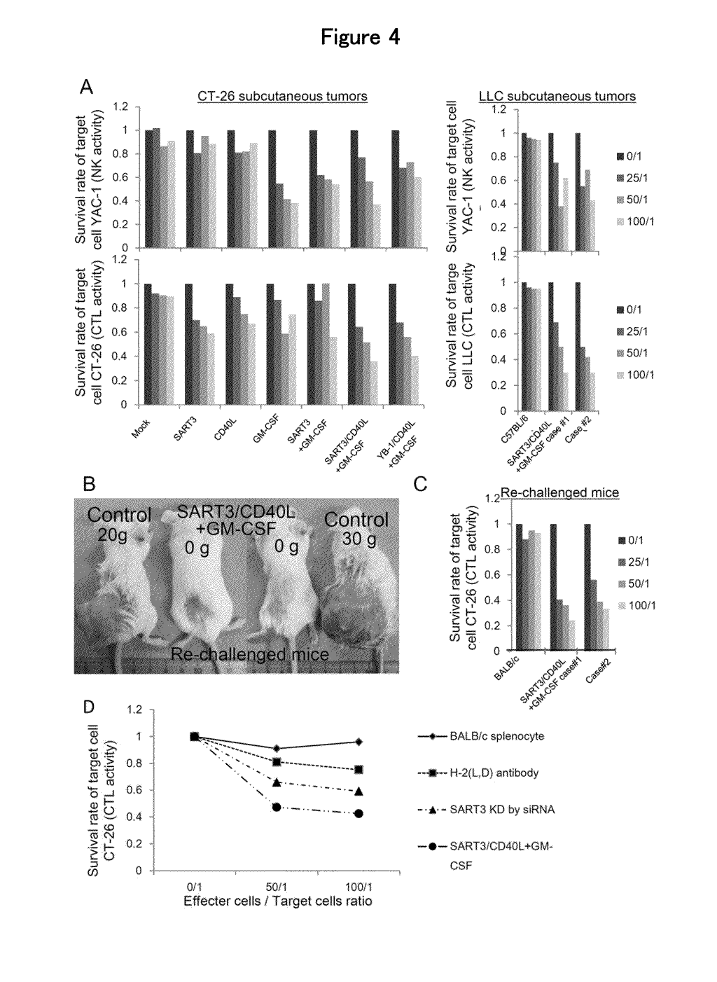

BALB/c and CB57/BL6 mice have normal immune system, two mechanisms as for antitumor effect were hypothesized: innate and/or acquired immunity. At first, we explored the activity of NK cells, because the activation of innate immunity is prerequisite for the induction of acquire immunity. YAC-1 cells are originated from mouse lymphoma and known as highly susceptible to the killing by NK cells. None of the polyplex micelles encapsulating Mock, SART3 alone or CD40L alone increased the NK activity (FIG. 4A, left upper panel). On the other hands, the polyplex micelles composed with GM-CSF transgene, such as GM-CSF alone, GM-CSF+SART3 and GM-CSF+SART3/CD40L regimen, obviously upregulated the NK activity (FIG. 4A, left upper panel).

To evaluate the CTL activity, we selected the method of CFSE-based cytotoxicity assay using target cells of CT26 or LLC/3LL due to its high sensitivity. In CT26 subcutaneous tumor model (FIG. 4A, left bottom panel), the number of CFSE-labeled viable target CT26 cells was decreased upon the higher ratio of effector: splenocyte to the target cells for the DNA vaccine treatment with SART3, CD40L and GM-CSF combination genes, but did not remarkably changed for the mock control, GM-CSF alone or GM-CSF+SART3 group (FIG. 4A, left bottom panel). In LLC/3LL subcutaneous tumor model (FIG. 4A, right bottom panel), the number of CFSE-labeled viable target LLC/3LL cells was decreased for the DNA vaccine group in an effector/target cell ratio, but not for the control (FIG. 4A, right bottom panel). BALB/c mice have MHC haplotype “d”, while C57BL/6 mice have haplotype “b”. These results suggest our DNA vaccine has advantages to omit the identification of effective epitopes and to use whole sequence of tumor specific antigen, and may be able to adopt the various MHC haplotype.

YB-1 Loading-DNA Vaccine Represents this Vaccine Platform's Usefulness to Induce CTL Activation and Anti-Tumor Effect.

To examine whether the DNA vaccine platform is able to work with other TAAs, we administered the DNA vaccine encapsulating YB-1, CD40L and GM-CSF combination to the mice harboring CT26 peritoneal dissemination. As similarly as the SART3-loading DNA vaccine, the YB-1-loading DNA vaccine significantly elongated the survival (47.2±12.8 days) more than the mock control (32.5±9.8 days), and the Kaplan-Meier analysis represents a significant increase in survival for the YB-1-loading DNA vaccine compared with the mock control (P=0.02; FIG. 2B left panel). Furthermore, there were elicited for high CTL and NK activities as similarly as the SART3-loading DNA vaccine (FIG. 4A).

### Re-Challenge Experiment Represents the Acquired Rejection Memory Immunity by the DNA Vaccine Treatment.

In CT26 peritoneal dissemination model, long-period survivors were appeared only in the mice receiving the DNA vaccine with SART3, CD40L and GM-CSF genes. To elucidate whether the DNA vaccine elicits CT26 specific rejection memory immunity, the CT26 re-challenge (1×10⁶ cell) was carried out in the long-term survivors and compared with the non-vaccinated control. As shown in FIG. 4B, the re-challenged CT26 cancer was rejected completely for the DNA vaccine group (all eight cases), but subcutaneous tumors were formed for the control mice. Upon the mechanism for the CT26 rejection, the CTL activity for the mice receiving the DNA vaccine was increased in an effector/target cell ratio-dependent manner (FIG. 4C) besides the NK activity (data not shown). On the other hands, CTL or NK activity was neither changed for the control mice (FIG. 4C).

### Specificity of Cell Killing Activity of CTL to the TAA and MHC Molecules.

To examine the MHC-restriction of CTL activity, we verify the MHC-mediated CTL activity using MHC (H-2L and H-2D) blocking antibodies (FIG. 4D). The CTL activity of splenocytes from mice receiving the DNA vaccine with SART3, CD40L and GM-CSF combination was remarkably declined to one-third of the control values under MHC blocking condition. To examine the TAA-specificity of CTL activity, we knocked down SART3 expression in CT26 cells using SART3-targeting siRNA and confirmed the mRNA expression down to 30% of the siRNA control. The CTL activity of splenocytes from the DNA vaccine-treated mice against the SART3 silencing CT26 cells was decreased compared with the non-treated control (FIG. 4D), despite the loss of CTL activity was not much as the MHC blocking due to the remained SART3 expression. These results suggest that the CTL activity elicited by the DNA vaccine was mediated through the SART3 peptides on the MHC class 1 molecule.

Immunohistochemistry Reveals that the Infiltration of GM-CSF, CD11c, CD4 and/or CD8a-Positive Immune Cells into Lymph Nodes, Spleen and Tumors is Increased for the DNA Vaccine Treatment

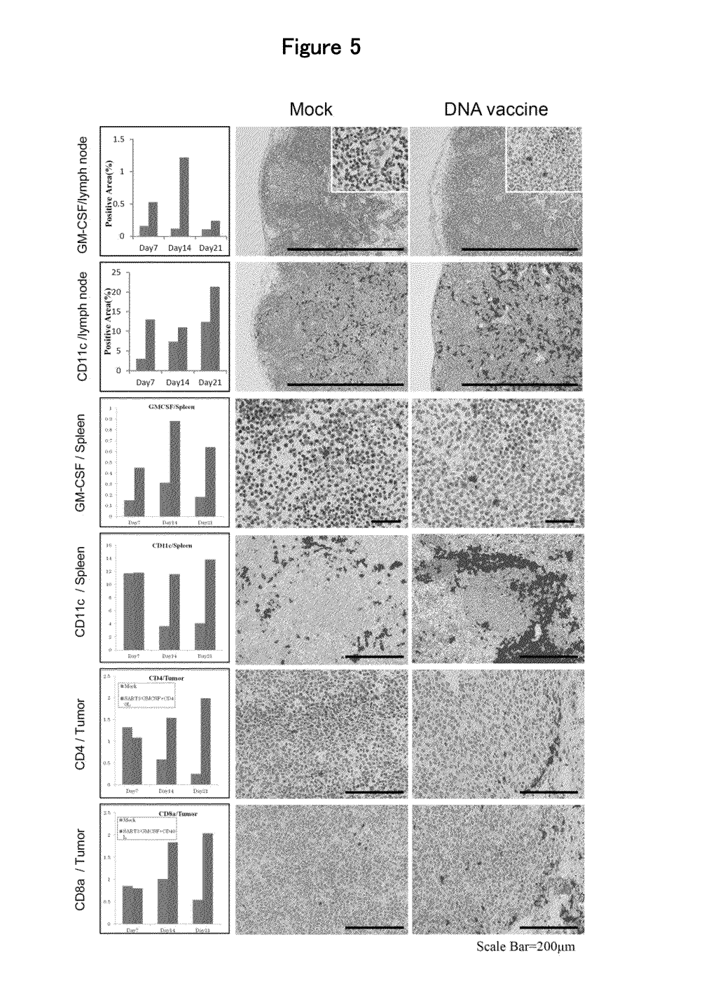

The immunohistochemistry clarified the changes in infiltration of immune cells expressing GM-CSF, CD11c, CD4 and CD8a in lymph nodes, spleen and tumor tissues (FIG. 5). Except in spleen on day 7 after the DNA vaccination, the several-fold increases in GM-CSF and CD11c expression were observed in lymph nodes and spleen from day 7 to day 21 for the DNA vaccine group compared with the control. As for CD4- and CD8a-expressions in tumor tissues, there were not significant differences between the DNA vaccine and the mock control at the early phase (day 7) after the treatment. Thereafter, the increases in CD4- and CD8a-positive cells were depicted for the DNA vaccine group but not for the control group on day 14 (right panel pictures) and day 21. The quantitation analysis (left panel) confirms that the expression levels of CD4 and CD8a in tumors were 3-10-fold higher for the DNA vaccine group than the control on days 14 and 21 after the vaccination.

### Validation of MUC1 and Survivine as TAA for Gene Vaccine

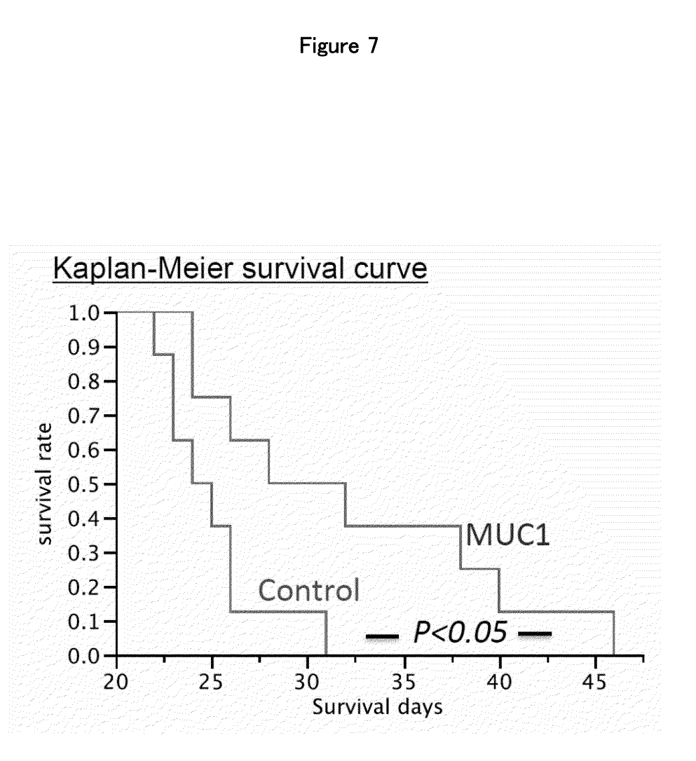

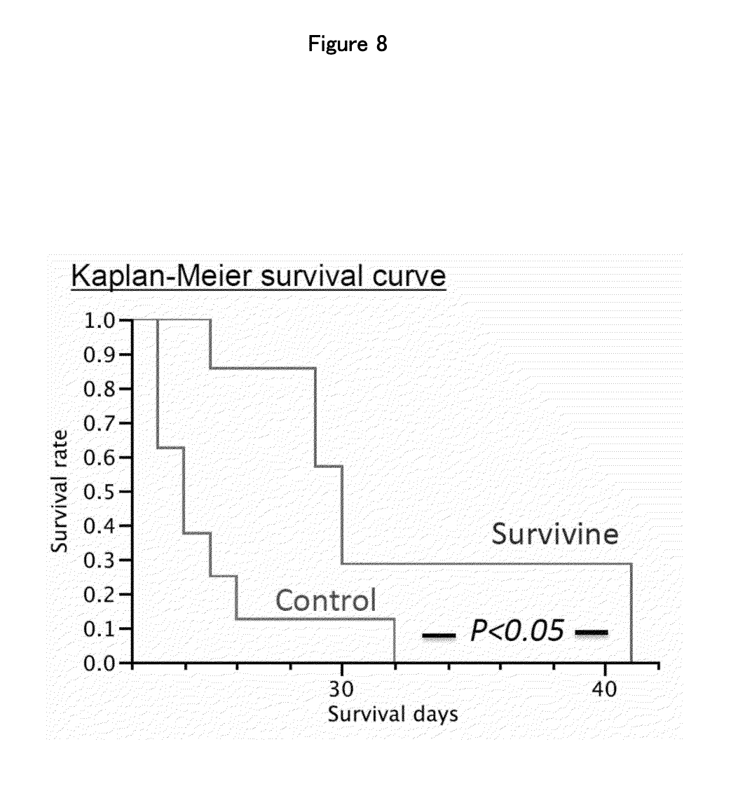

Both DNA vaccines loaded with MUC1 or survivine TAA gene significantly elongated the survival period more than the mock control (32.3±8.2 vs 24.9±3.1 days; 32.4±6.8 vs 25.0±3.0 days, respectively). The Kaplan-Meier analysis shows the survival rates were significantly improved for both MUC1- and survivine-loading DNA vaccines (log-lank test: P<0.05 in FIG. 7 and FIG. 8), suggesting that MUC1 and survivine are effective TAA for DNA vaccine.

### Chimera of Single Chain of Variable Fragment of Anti-CD28 Antibody Fused to CD86 Molecule has an Adjuvant Effect

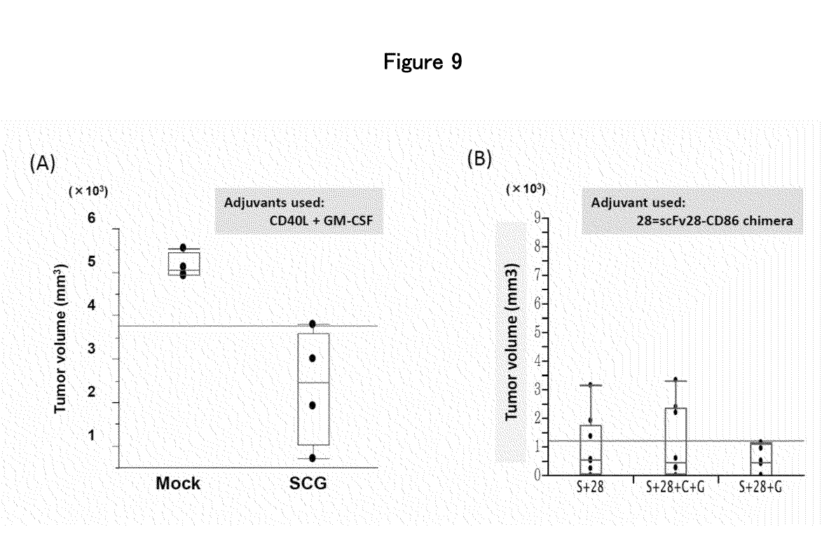

The tumor weights were significantly lower for SART3/scFv28-CD86, SART3/scFv28-CD86/GM-CSF and SART3/scFv28-CD86/GM-CSF/CD40L-loading DNA vaccines than SART3/GM-CSF/CD40L or mock control group (0.92±0.1 (median 0.55) g; 0.59±0.1 (median 0.51) g; 1.2±0.9 (median 0.55) g versus 2.4±0.3 (median 2.5) g; 5.2±0.2 (median 5.0) g, respectively in FIG. 9). These results suggest that scFv28-CD86 chimera gene exhibits an adjuvant effect on DNA vaccine.

### Discussion

In the present study, we have constructed a novel DNA vaccine. In peritoneal dissemination mice model, the DNA vaccine loaded with tumor-associated antigen (TAA) of SART3 or YB-1 gene plus CD40L and GM-CSF adjuvant genes exerted the survival elongation with the burst of CTL activity and completely rejected the re-challenged tumor cells, suggesting the acquirement of tumor-specific rejection immunity. In subcutaneous tumor models, the DNA vaccine regimen induced high CTL activities and the infiltration of CD4- and CD8a-positive T-lymphocytes into subcutaneous tumors and distant lung organ, of which cells depletion ameliorated the anti-tumor efficacy of the DNA vaccine. These results indicate the micelle loaded with TAA, CD40L and GM-CSF combination exhibits a high potential for DNA vaccine effect to elicit specific anti-tumor immunity.

To sensitize the weak immunogenicity of TAA, complete and/or incomplete Fleund's adjuvants are co-injected with peptide vaccines [ref 25]. For cell vaccines, viral and bacterial components, such as pCpG motif, may work as adjuvants [ref 26] and DC cell itself has high potential of antigen-presentation [ref 1]. For gene vaccines, it has been explored on the methods of adjuvant effect, such as polyubquitination sequence [ref 27] and heat-shock proteins for scavenger molecules [ref 28], to resolve the weak antigenicity issues. In this study, we tried the approach for combined expression of TAA, cytokine and co-stimulatory factor using micelle-based gene carriers. Several clinical trials for cell vaccine have reported that the transduction of cytokine GM-CSF or co-stimulatory molecule CD40L up-regulates the antigen-presentation [ref 29, 30]. Therefore, we initially assessed the micelle encapsulating single TAA, CD40L or GM-CSF gene, but failed to suppress tumor growth (FIG. 2D) or prolong the survival (FIG. 2B right panel; Table 3). On the other hands, the triple combination of TAA, CD40L and GM-CSF induced the cure of dissemination in 40% of the DNA vaccine-treated mice (FIG. 2B left panel) and protected lung metastasis (FIG. 3). A simple method of vaccination, for the first time, is accomplished by i.p. administration of TAA/CD40L/GM-CSF-loading micelles.

We tested two genes overexpressing in a variety of cancers as a candidate of TAAs in this study. SART3 has been reported the sequences of epitope-peptides with vaccination effect [ref 31]. Although the potential of epitope-peptides of YB-1 remains unclear, the possibility of YB-1's antigenicity was reported by SEREX analysis in patients with neuroblastoma [ref 32]. Transduction of TAA genes in vivo leads to the intracellular events that TAA-gene's coding proteins are expressed in the cytoplasmic region, degraded to the fragmented peptides in endosomes, and exposed on various types of MHC molecules. In tumor models of both BALB/c and CB57/BL6 mouse strains, the anti-tumor efficacy via CTL activation was induced by our DNA vaccine regimen, suggesting transduced SART3 and YB-1 antigens could exhibit high immunogenicity due to multiple species of epitope-peptides bound on different MHC haplotypes. Furthermore, recent technologies in genome-wide microarray and sequencing enable the screening of many candidate genes for TAA [ref 33]. Therefore, the screening of TAA genes is more systemically possible for gene vaccine compared with peptide vaccine and that all patients are eligible for gene vaccine regardless of the MHC haplotypes.

The nano-sized carrier device has a property to adsorb into lymphatic vessels after i.p. administration [ref 34]. For instance, ultrasound-responsive liposome surrounded with mannose-ligands, which is up-taken up the reticulo-endothelial system (e.g. spleen), releases the transgenes when the liposome is relapsed by ultrasound stimulation [ref 35]. The block/homo polyplex micelles also exhibit the characteristics to delivery to lymph nodes and spleen predominantly after i.p. administration, as previously demonstrated [ref 24]. Subsequently, some of micelles seemed to be up-taken into DC cells (FIG. 1), where the coordination of GM-CSF and CD40L may break out the energy status of TAA immunogenicity in DC cells. This is supported by the immunohistochemical analysis that the GM-CSF and CD11c-positive immune cells were increased in lymph nodes and spleen at early time-point (FIG. 5) after the micelle administration. The transduced GM-CSF may not only maturate DC cells but also stimulate NK cells, because the treatment groups without GM-CSF did not activate the NK activity (FIG. 4A). Under the activated condition of innate immunity, dual TAA/MHC class-1 and -2 and CD40/CD40L signals in DC cells might transmit the activation signal to CD8 and CD4-lymphocytes, respectively. The complete rejection of re-challenged tumor cells indicate our DNA vaccine regimen elicited the specific-rejection memory immunity, which was supported by the increase and infiltration of helper (CD4+) and cytotoxic (CD8a+) T-lymphocytes in tumor tissues (FIG. 5).

In this study, we designed the vaccination protocols mimicking the clinical settings of adjuvant therapy after surgical resection. The tumor microenvironments shift to the immune-suppressive balance, where regulatory T-cell (Treg) and myeloid-derived suppressor cells (MDSC) are increased [ref 36, 37], in accordance with the cancer progression, although our preliminary experiments showed no increase in Treg cells until one week after the implantation of cancer cells (data not shown).

### REFERENCES

25. Slingluff C L, Petroni G R, Smolkin M E, Chianese-Bullock K A, Smith K, Murphy C, et al. Immunogenicity for CD8+ and CD4+ T cells of 2 formulations of an incomplete freund's adjuvant for multipeptide melanoma vaccines. J Immunother. 2010; 33:630-8.
26. Kuwajima S, Sato T, Ishida K, Tada H, Tezuka H, Ohteki T. Interleukin 15-dependent crosstalk between conventional and plasmacytoid dendritic cells is essential for CpG-induced immune activation. Nature immunology. 2006; 7:740-6.
27. Zhang M, Obata C, Hisaeda H, Ishii K, Murata S, Chiba T, et al. A novel DNA vaccine based on ubiquitin-proteasome pathway targeting ‘self’-antigens expressed in melanoma/melanocyte. Gene therapy. 2005; 12:1049-57.
28. Ciupitu A M, Petersson M, O'Donnell C L, Williams K, Jindal S, Kiessling R, et al. Immunization with a lymphocytic choriomeningitis virus peptide mixed with heat shock protein 70 results in protective antiviral immunity and specific cytotoxic T lymphocytes. The Journal of experimental medicine. 1998; 187:685-91.
29. Zarei S, Schwenter F, Luy P, Aurrand-Lions M, Morel P, Kopf M, et al. Role of GM-CSF signaling in cell-based tumor immunization. Blood. 2009; 113:6658-68.
30. Ma D Y, Clark E A. The role of CD40 and CD154/CD40L in dendritic cells. Seminars in immunology. 2009; 21:265-72.
31. Yang D, Nakao M, Shichijo S, Sasatomi T, Takasu H, Matsumoto H, et al. Identification of a gene coding for a protein possessing shared tumor epitopes capable of inducing HLA-A24-restricted cytotoxic T lymphocytes in cancer patients. Cancer research. 1999; 59:4056-63.
32. Zheng J, Jing W, Orentas R J. Discovery of YB-1 as a new immunological target in neuroblastoma by vaccination in the context of regulatory T cell blockade. Acta biochimica et biophysica Sinica. 2009; 41:980-90.
33. Imai K, Hirata S, Irie A, Senju S, Ikuta Y, Yokomine K, Harao M, Inoue M, Tsunoda T, Nakatsuru S, Nakagawa H, Nakamura Y, Baba H, Nishimura Y: Identification of a novel tumor-associated antigen, cadherin 3/P-cadherin, as a possible target for immunotherapy of pancreatic, gastric, and colorectal cancers. Clin Cancer Res. 2008 Oct. 15; 14(20):6487-95.
34. Hirano K, Hunt C A. Lymphatic transport of liposome-encapsulated agents: effects of liposome size following intraperitoneal administration. Journal of pharmaceutical sciences. 1985; 74:915-21.
35. Un K, Kawakami S, Suzuki R, Maruyama K, Yamashita F, Hashida M. Enhanced transfection efficiency into macrophages and dendritic cells by a combination method using mannosylated lipoplexes and bubble liposomes with ultrasound exposure. Human gene therapy. 2010; 21:65-74.
36. Clark C E, Hingorani S R, Mick R, Combs C, Tuveson D A, Vonderheide R H. Dynamics of the immune reaction to pancreatic cancer from inception to invasion. Cancer research. 2007; 67:9518-27.
37. Pan P Y, Wang G X, Yin B, Ozao J, Ku T, Divino C M, et al. Reversion of immune tolerance in advanced malignancy: modulation of myeloid-derived suppressor cell development by blockade of stem-cell factor function. Blood. 2008; 111:219-28.

### Figure Legends

### FIG. 1 Polyplex Micelle Distribution and Gene Expression in Vivo.

(A) Fluolid-labeled polyplex micelles with pDNA (50 ug; N/P ratio=10) were administered in the peritoneal cavity of mice. Twenty-four hour later, organ tissues were snap-frozen to examine the tissue distribution under fluorescence laser confocal microscopy. The sections were also immunostained with an anti-CD11c antibody to examine the co-localization of polyplex micelles and dendritic cells. The polyplex micelles were mainly localized in spleen (left panel) and lymph nodes (center panel), and the merge imaging shows the co-localization of polyplex micelles and dendritic cells in lymph nodes (right panel). (B) Total RNA was extracted from the frozen tissues at 24 hours after i.p. administration of polyplex micelles with GM-CSF gene, and the resultant cDNA samples were subjected to real-time RT-PCR analysis. The expression of GM-CSF was predominantly up-regulated in spleen and lymph nodes, and detected much less in lung and other organs.

### FIG. 2 Anti-Tumor Efficacy of Polyplex Micelle-Based DNA Vaccine in Mice Harboring Peritoneal Dissemination and Subcutaneous Tumors.

(A) The scheme shows the vaccination schedule with polyplex micelle encapsulating therapeutic genes (Table 3) in CT26 peritoneal dissemination model. (B) The Kaplan-Meier survival curve demonstrates that the DNA vaccine encapsulating SART3, CD40L and GM-CSF significantly elongated the survival for mice bearing CT26 dissemination compared with the mock control (left panel). No significant improvement in survival rates was detected for the groups with single gene transduction (right panel). (C) The scheme shows the vaccination schedule with the polyplex micelle encapsulating the therapeutic genes in subcutaneous tumor models of CT26 and LLC. (D) The tumor weight of CT26 cancer on day 14 was significantly less for the DNA vaccine group than the mock control or each single gene treatment (left panel). In LLC subcutaneous tumors, it significantly decreased for the DNA vaccine group compared with the mock control or single gene treatment (right panel).

### FIG. 3 Protective Effect of Polyplex Micelle-Based DNA Vaccine on Lung Metastasis of LLC Tumors.

(A) Lung tissues were obtained from the mice with the indicated DNA vaccine or mock on day 28 after subcutaneous inoculation of LLC cancer. H&E staining shows that lung metastasis was highly developed in the mock control (4/4 cases; left panel), whereas that was not detected in the DNA vaccine group (0/4 cases; right panel). (B) Immunohistochemistry demonstrates that the infiltration of CD4- and CD8a-positive T lymphocytes into the lung tissues were up-regulated (P<0.05 and P<0.01, respectively) for the DNA vaccine group (right panel) compared with the mock control (left panel).

### FIG. 4 Upregulation in NK and CTL Activities and Acquirement of TAA-Specific Rejection Memory Immunity by Polyplex Micelle-Based DNA Vaccine.

(A) Splenocytes (effector cells) were isolated from mice bearing CT26 and LLC subcutaneous tumors, and consequently co-incubated with irradiated CSFE-labeled CT26 or YAC-1 (target cells) at the indicated effector/target cell ratio, followed by the CTL or NK assay through flow-cytometry, respectively. The NK activity (upper panel) was increased in the all treatment groups with GM-CSF transgene. In contrast, the CTL activity (lower panel) was up-regulated only in the polyplex micelle-encapsulating SART3 or YB-1, CD40L and GM-CSF (DNA vaccine group) in an effector/target cell ratio-dependent manner. (B) CT26 cells were re-challenged at the flank region in the mice survived more than 80 days, and the formation of subcutaneous tumors were monitored for another 60 days. The specific rejection immunity was gained in mice with only the DNA vaccine group, but not in the controls. (C) Splenocytes were isolated after the re-challenge of CT26 as shown in FIG. 2A, and co-incubated with the CFSE-labeled target CT26 cells. The CTL activity for long-term survivor mice received the DNA vaccine was increased, but not the control mice without the DNA vaccine. (D) The blocking experiments using ant-MHC class 1 (H-2L and -2D) antibodies or SART3 knockdown by siRNA transfection in CTL assay confirmed the specificity of CFSE-target cell killing to MHC and TAA species.

FIG. 5 Immunohistochemical Analysis of Immune Cells Infiltrating into Tumor and Immune Organ Tissues.

Tissue sections from spleen, lymph nodes and tumors were immunostained with the indicated antibodies. The protein signals were digitalized (red color in right panel) above certain threshold level. The expression levels of protein signals are quantitated by the strength of digitalized signals in accordance with the NIS-Element program (left panel).

### FIG. 6 Liposome-Based DNA Vaccine Encapsulating SART3, CD40L and GM-CSF, and Subcutaneous Administration of DNA Vaccine in the Groin Region.

(A) pDNAs of SART3, CD40L and GM-CSF (total 50 ug) were encapsulated with liposome (Coatsome EL-01-C, NOF corp.) in accordance with the manufacture's protocol. The liposome-based DNA vaccine was intraperitoneally administered in mice harboring CT26 peritoneal dissemination, as similarly as the polyplex micelle-based DNA vaccine. The liposome-based DNA vaccine prolonged the survival (48.0±19.5 days) compared with the mock control (32.5±9.8 days; P=0.06 for log-rank test). (B) pDNAs of SART3, CD40L and GM-CSF (total 50 ug) were encapsulated with PEG-b-[Pasp(DET)]/Pasp(DET) at 10 of N/P ratio. The polyplex micelle-based DNA vaccine was subcutaneously administered in the groin region of mice harboring CT26 peritoneal dissemination. Left panel demonstrates that the polyplex micelle-based DNA vaccine prolonged the survival compared with the mock and saline controls (P=0.02 and P=0.005, respectively, for log-rank test). Right panel shows that the Fluolid-labeled polyplex micelles were distributed into lymph nodes at the groin region in the mice.

### FIG. 7 Kaplan-Meier Survival Curve

CT26 colon cancer cells were implanted into the peritoneal cavity of BALB/c mice. One week later, a polyplex micelle with mouse MUC1/CD40L/GM-CSF genes was intraperitoneally administered, and then the survival of mice was monitored. The Kaplan-Meier analysis shows the survival rates were significantly improved for both MUC1- and survivine-loading DNA vaccines (log-lank test: P<0.05), suggesting that MUC1 and survivine are effective TAA for DNA vaccine.

### FIG. 8 Kaplan-Meier Survival Curve

CT26 colon cancer cells were implanted into the peritoneal cavity of BALB/c mice. One week later, a polyplex micelle with mouse survivine/CD40L/GM-CSF genes was intraperitoneally administered, and then the survival of mice was monitored. The Kaplan-Meier analysis shows the survival rates were significantly improved for both MUC1- and survivine-loading DNA vaccines (log-lank test: P<0.05), suggesting that MUC1 and survivine are effective TAA for DNA vaccine.

### FIG. 9 CT26 Subcutaneous Tumor

CT26 colon cancer cells were subcutaneously implanted in flank region, and one day later a block/homo mixed polyplex micelle encapsulating with SART3 plus indicated adjuvant genes (60 ug of pDNA, NP ratio=10) was administered into the peritoneal cavity of mice: (A) adjuvants=CD40L+GM-CSF; and (B) adjuvant=“28=scFv28-CD86 chimera”. The tumor weights were significantly lower for SART3/scFv28-CD86, SART3/scFv28-CD86/GM-CSF and SART3/scFv28-CD86/GM-CSF/CD40L-loading DNA vaccines than SART3/GM-CSF/CD40L or mock control group (0.92±0.1 (median 0.55) g; 0.59±0.1 (median 0.51) g; 1.2±0.9 (median 0.55) g versus 2.4±0.3 (median 2.5) g; 5.2±0.2 (median 5.0) g, respectively).

### INDUSTRIAL APPLICABILITY

The present data have revealed the potential of micelle-based gene therapy comprising of TAA (SART3 or YB-1), CD40L and GM-CSF combination as a DNA vaccine in mouse tumor models. The DNA vaccine prolonged the survival for mice harboring peritoneal dissemination and inhibited the growth and metastasis of subcutaneous tumors with the burst of CTL activation and the infiltration of CD4- and CD8a-positive lymphocytes (CTL) into tumors. It is concluded that TAA/CD40L/GM-CSF-loading micelle is a novel DNA vaccine platform to elicit the anti-tumor immunity against intractable cancers.

1-21. (canceled)

22. A pharmaceutical composition for treating a tumor, which is a micelle encapsulating at least one tumor-associated antigen gene and at least one adjuvant gene.

23. The pharmaceutical composition of claim 22, wherein the tumor-associated antigen gene is at least one selected from the group consisting of squamous cell carcinoma antigen recognized by T cells 3 (SART3), Y-box binding protein 1 (YB-1), Mucin 1, cell surface associated (MUC1) and Survivin.

24. The pharmaceutical composition of claim 22 or 23, wherein the adjuvant gene is at least one selected from the group consisting of Granulocyte-macrophage colony-stimulating factor (GM-CSF) and CD40L.

25. The pharmaceutical composition according to claim 22 or 23, wherein the adjuvant gene is any one of polynucleotide selected from the group consisting of (a) to (e) below:
(a) a polynucleotide comprising the nucleotide sequence of SEQ ID NO: 13;
(b) a polynucleotide encoding a protein consisting of the amino acid sequence of SEQ ID NO: 14;
(c) a polynucleotide encoding a protein consisting of an amino acid sequence wherein 1 to 40 amino acids are deleted, substituted, inserted and/or added in the amino acid sequence of SEQ ID NO: 14, and having an activity of 28scFv(LH)-CD86 chimera;
(d) a polynucleotide encoding a protein having an amino acid sequence having at least 85% homology to the amino acid sequence of SEQ ID NO: 14, and having an activity of 28scFv(LH)-CD86 chimera; and,
(e) a polynucleotide which hybridizes to a polynucleotide consisting of a nucleotide sequence complementary to the nucleotide sequence of SEQ ID NO: 13 under stringent conditions, and which encodes a protein having an activity of 28scFv(LH)-CD86 chimera.

26. The pharmaceutical composition of claim 25, comprising the polynucleotide in combination with any one or both of GM-CSF and CD40L.

27. The pharmaceutical composition according to claim 22, wherein the micelle is a polyion complex micelle.

28. The pharmaceutical composition according to claim 22 wherein the tumor is one selected from the group consisting of osteosarcoma, soft tissue sarcoma, carcinoma of the breast, carcinoma of the lung, carcinoma of the bladder, carcinoma of the thyroid gland, carcinoma of the prostate, carcinoma of the colon, colorectal carcinoma, carcinoma of the pancreas, carcinoma of the stomach, carcinoma of the liver, carcinoma of the uterus, carcinoma of the cervix, carcinoma of the ovary, Hodgkin lymphoma, non-Hodgkin lymphoma, neuroblastomas, melanomas, myelomas, Wilms tumors, acute myelocytic leukemia (AML), chronic myelocytic leukemia (CML), acute lymphocytic leukemia (ALL), chronic lymphocytic leukemia (CLL), gliomas, and retinoblastomas.

29. A method for preventing and/or treating a tumor in a subject, comprising administering an effective amount of a micelle encapsulating at least one tumor-associated antigen gene and at least one adjuvant gene to the subject.

30. The method according to claim 29, wherein the tumor is prevented by acquired rejection memory immunity.

31. The method according to claim 29 or 30, wherein the tumor-associated antigen gene is at least one selected from the group consisting of squamous cell carcinoma antigen recognized by T cells 3 (SART3), Y-box binding protein 1 (YB-1), Mucin 1, cell surface associated (MUC1), and Survivin.

32. The method according to claim 29, wherein the adjuvant gene is at least one selected from the group consisting of Granulocyte-macrophage colony-stimulating factor (GM-CSF) and CD40L.

33. The method of according to claim 29, wherein the adjuvant gene is any one of polynucleotide selected from the group consisting of (a) to (e) below:
(a) a polynucleotide comprising the nucleotide sequence of SEQ ID NO: 13;
(b) a polynucleotide encoding a protein consisting of the amino acid sequence of SEQ ID NO: 14;
(c) a polynucleotide encoding a protein consisting of an amino acid sequence wherein 1 to 40 amino acids are deleted, substituted, inserted and/or added in the amino acid sequence of SEQ ID NO: 14, and having an activity of 28scFv(LH)-CD86 chimera;
(d) a polynucleotide encoding a protein having an amino acid sequence having at least 85% homology to the amino acid sequence of SEQ ID NO: 14, and having an activity of 28scFv(LH)-CD86 chimera; and,
(e) a polynucleotide which hybridizes to a polynucleotide consisting of a nucleotide sequence complementary to the nucleotide sequence of SEQ ID NO: 13 under stringent conditions, and which encodes a protein having an activity of 28scFv(LH)-CD86 chimera.

34. The method according to claim 33, wherein said polynucleotide may be used in combination with any one or both of GM-CSF and CD40L.

35. The method according to claim 29, wherein the micelle is a polyion complex micelle.

36. The method according to claim 29, wherein the tumor is one selected from the group consisting of osteosarcoma, soft tissue sarcoma, carcinoma of the breast, carcinoma of the lung, carcinoma of the bladder, carcinoma of the thyroid gland, carcinoma of the prostate, carcinoma of the colon, colorectal carcinoma, carcinoma of the pancreas, carcinoma of the stomach, carcinoma of the liver, carcinoma of the uterus, carcinoma of the cervix, carcinoma of the ovary, Hodgkin lymphoma, non-Hodgkin lymphoma, neuroblastomas, melanomas, myelomas, Wilms tumors, acute myelocytic leukemia (AML), chronic myelocytic leukemia (CML), acute lymphocytic leukemia (ALL), chronic lymphocytic leukemia (CLL), gliomas, and retinoblastomas.

37. A pharmaceutical composition for treating a tumor, which is a micelle encapsulating at least one tumor-associated antigen gene and adjuvant gene, wherein the adjuvant gene comprises both Granulocyte-macrophage colony-stimulating factor (GM-CSF) and CD40L.

38. The pharmaceutical composition of claim 37, wherein the tumor-associated antigen is at least one selected from the group consisting of squamous cell carcinoma antigen recognized by T cells 3 (SART3), Y-box binding protein 1 (YB-1), Mucin 1, cell surface associated (MUC1) and Survivin.

39. The pharmaceutical composition of claim 37, wherein the tumor-associated antigen is squamous cell carcinoma antigen recognized by T cells 3 (SART3).

40. The pharmaceutical composition of claim 37, wherein the tumor-associated antigen is Mucin 1, cell surface associated (MUC1).

41. The pharmaceutical composition according to any one of claims 37 to 40, wherein the adjuvant gene is any one of polynucleotide selected from the group consisting of (a) to (d) below:
(a) a polynucleotide comprising the nucleotide sequence of SEQ ID NO: 13;
(b) a polynucleotide encoding a protein consisting of the amino acid sequence of SEQ ID NO: 14;
(c) a polynucleotide encoding a protein having an amino acid sequence having at least 90% homology to the amino acid sequence of SEQ ID NO: 14, and having an activity of 28scFv(LH)-CD86 chimera; and,
(d) a polynucleotide comprising a nucleotide sequence having at least 90% homology to the nucleotide sequence of SEQ ID NO: 13, and which encodes a protein having an activity of 28scFv(LH)-CD86 chimera.

42. The pharmaceutical composition according to claim 37, wherein the micelle is a polyion complex micelle.
